(19) United States
(12) Patent Application Publication
(10) Pub. No.: US 2007/0129319 A1
(43) Pub. Date: Jun. 7, 2007

(54) Genes encoding human deubiquitinating enzymes

(75) Inventors: James Burrows, Belfast (GB); Michael McGrattan, Bangor (GB); James Johnston, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Queen's University of Belfast, Belfast (GB)

(21) Appl. No.: 10/579,169

(22) PCT Filed: Nov. 12, 2004
(86) PCT No.: PCT/GB04/04820

§ 371 Date: Jun. 14, 2006

(30) Foreign Application Priority Data
Nov. 14, 2003 (GB) 0326598.0
Apr. 17, 2004 (GB) 0408629.4

(51) Int. Cl. A61K 48/00; C12Q 1/68; G01N 33/574; C07H 21/04; C07K 14/82; C07K 16/30

(52) U.S. Cl. 514/44; 435/6; 435/7.23; 435/69.1; 435/320.1; 435/325; 530/350; 530/388.8; 536/23.5

(57) ABSTRACT

Described is a novel human deubiquitinating enzyme, DUB-3, (SEQ ID NO: 1), and variants and fragments thereof. Also described are nucleic acid molecules encoding the enzyme, and use of the polypeptide and nucleic acid molecules in treatment of cancer, assays.

### FIELD OF THE INVENTION

The present invention relates to a novel gene and protein and uses thereof in the treatment and detection of abnormal cells and cancer. Moreover, the invention relates to methods for the diagnosis of cancer, the treatment of cancer, and the development of therapeutic agents for the treatment of cancer.

### BACKGROUND TO THE INVENTION

It has become apparent that the ubiquitin proteasome pathway plays an important role in the regulation of many cellular processes such as the cell cycle¹, transcription², apoptosis³, receptor internalisation and vesicle trafficking^{4, 5}. This has led to attention being focused upon the proteins which modulate this pathway. These include a group of enzyme complexes, referred to as E3-ligases, as well as the E2 conjugating enzymes with which they interact to transfer ubiquitin to its targets⁶. Another large group of proteins involved in this process are the deubiquitinating enzymes, a family of ubiquitin specific proteases that cleave ubiquitin from ubiquitin-conjugated proteins and are thought to act at several points in the ubiquitin pathway including polyubiquitin precursor processing, the removal of ubiquitin from substrates to rescue them from degradation and the removal of residual ubiquitin to aid proteasomal degradation⁷.

These enzymes are classified into two main sub-families, the ubiquitin processing proteases (UBPs) and the ubiquitin carboxy-terminal hydrolases (UCHs) which are both cysteine proteases whose active site contain a cysteine, histidine and aspartate residue⁸. The UBPs vary greatly in size and structural complexity but all contain six conserved homology domains (DHI-DHVI). The UCHs are a family of small closely related proteins which lack these six domains⁷. In addition to these two main sub-families the JAMM family of metalloproteases⁹, as well as the OTU family of isopeptidases¹⁰, have recently been identified as deubiquitinating enzymes.

Although the substrates of most deubiquitinating enzymes are unknown, it is clear that they play important roles in the regulation of many cellular processes. For example, FAF (fat facets) can regulate drosophila eye development¹¹, FAM (fat facets in mice) (USP9) is vital for embryonic development in mice ¹², UBP3¹³ and D-ubp-64E¹⁴ play a role in transcriptional silencing and other UBPs have been shown to interact with important cell signalling proteins, such as p53¹⁵, Rb^{16, 17}, β-catenin¹⁸, BRCA1¹⁹, and VHL²⁰.

Deubiquitinating enzymes have also been implicated in cell transformation. In particular, a truncated isoform of the mammalian UBP, tre-2, which has no deubiquitinating activity, has been shown to transform 3T3 fibroblasts²¹. Also, over-expression of

unp (USP4), another mammalian UBP, induces transformation of NIH3T3 cells injected into athymic mice²² and elevated levels of its human orthologue, unph, have been found in small cell carcinomas and adenocarcinomas of the lung²³.

The DUB family of UBPs were identified in mice as haematopoietic specific deubiquitinating enzymes that are rapidly induced upon cytokine stimulation. DUB-1 is induced by IL-3, IL-5 and GM-CSF and is expressed in a number of haematopoietic cell types²⁴, whilst DUB-2 appears to be specifically regulated by IL-2 with its expression restricted to T-cells²⁵. DUB-2A has recently been added to this family and is also primarily expressed in haematopoietic cells²⁶ . All three of these genes are thought to form part of a head to tail repeat of DUB genes on mouse chromosome 7 that have resulted from a tandem duplication event²⁵. It has been suggested that the DUBs may play a role in the regulation of cell growth and survival. Both DUB-1 and DUB-2 are cytokine inducible immediate early genes^{24, 25} and high-level expression of DUB-1 results in cell cycle arrest prior to S-phase²⁷. Moreover, the inventors have recently shown that DUB-2 expression can markedly inhibit apoptosis induced by cytokine withdrawal²⁸.

Our previous work on DUB-2 originated in a study of the IL-2 pathway in cells transformed with the Human T-cell lymphotropic virus I (HTLV-1). HTLV-1 is the etiologic agent for adult T-cell leukemia (ATL)²⁹ and is often associated with constitutive activation of the IL-2 signaling pathways^{30, 31}. Despite the active IL-2 signalling pathway, growth inhibitory gene products such as CIS and SOCS3 were not expressed. However, an antibody raised against murine DUB-2 cross reacted with a band constitutively expressed in these HTLV-1-transformed T-cell lines²⁸. When expressed in Ba/F3 cells DUB-2 was shown to markedly inhibit apoptosis induced by the withdrawal of cytokine²⁸ as well as prolonging STAT5 phosphorylation. This indicated that DUB-2 expression could influence cell survival possibly by modulating STAT5 activation. Interestingly, constitutive STAT activation plays an important role in many haematological malignancies including HTLV-1 dependent T-cell leukemia, Burkitt's lymphoma and myeloma, as well as a range of solid tumours. STAT activation is also required for the action of a range of oncogenes including src, ret and lck^{32, 33}.

Although DUB enzymes have been identified in mice, to date no DUB gene has been identified in humans.

### SUMMARY OF THE INVENTION

As described herein, the present inventors have now identified a novel human member of the DUB family of deubiquitinating enzymes and have shown its expression at the mRNA level in a range of tissues and cell lines. In addition the inventors demonstrate that the enzyme, (herein referred to as DUB-3, an active deubiquitinating enzyme, is induced at both the mRNA and protein level in response to IL-4 stimulation. Furthermore, the inventors have demonstrated that DUB-3 constitutive expression blocks proliferation and leads to an increase in the number of apoptotic cells, suggesting that its expression can influence both cell proliferation and survival. Moreover, the inventors have further identified a number of DUB-3 analogues (herein referred to as DUB-4, DUB-5, DUB-6, DUB-7, DUB-8 DUB-9, DUB-10, DUB-11 and DUB-12), which are also encompassed by the invention.

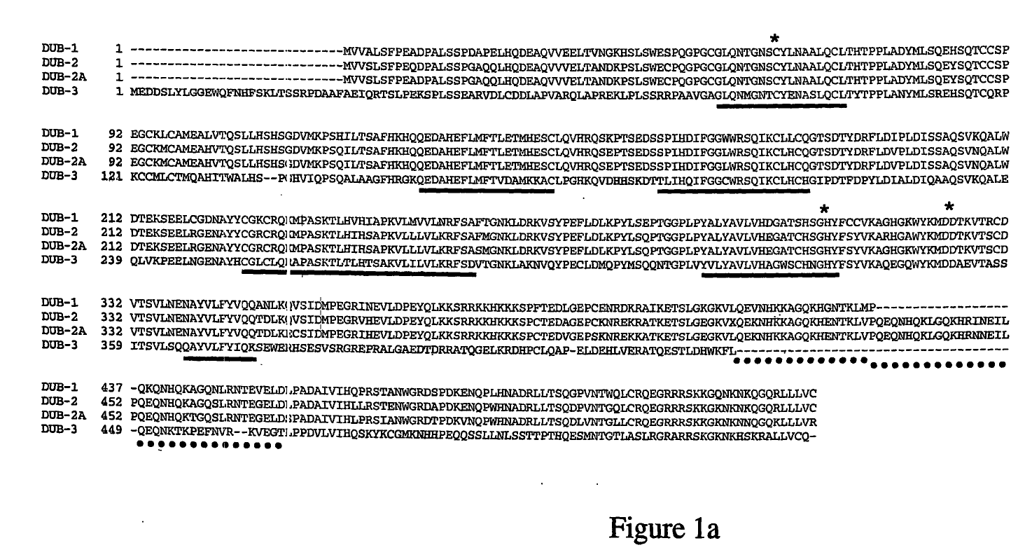

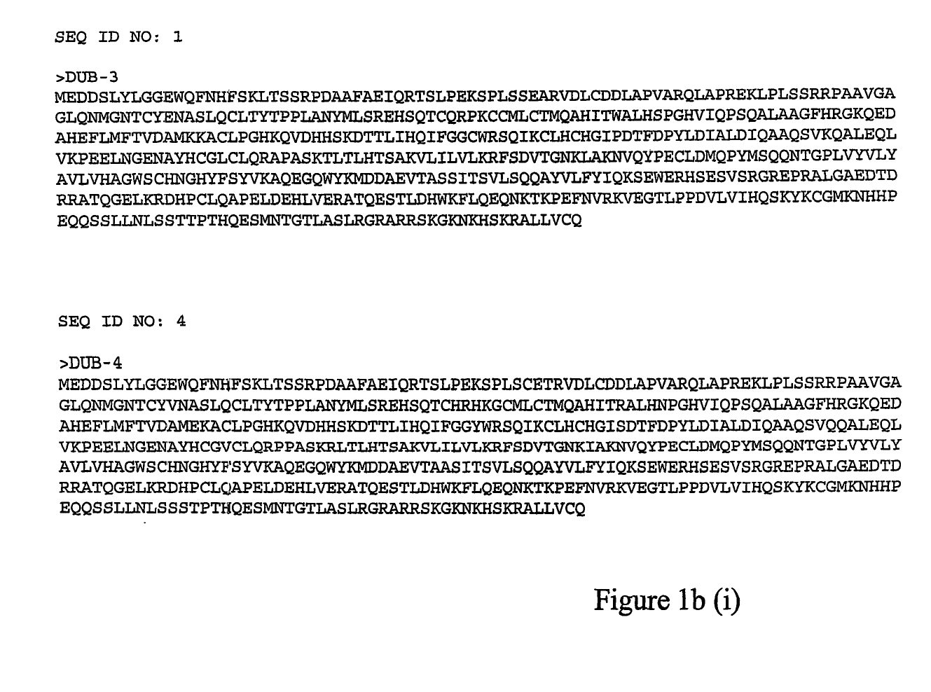

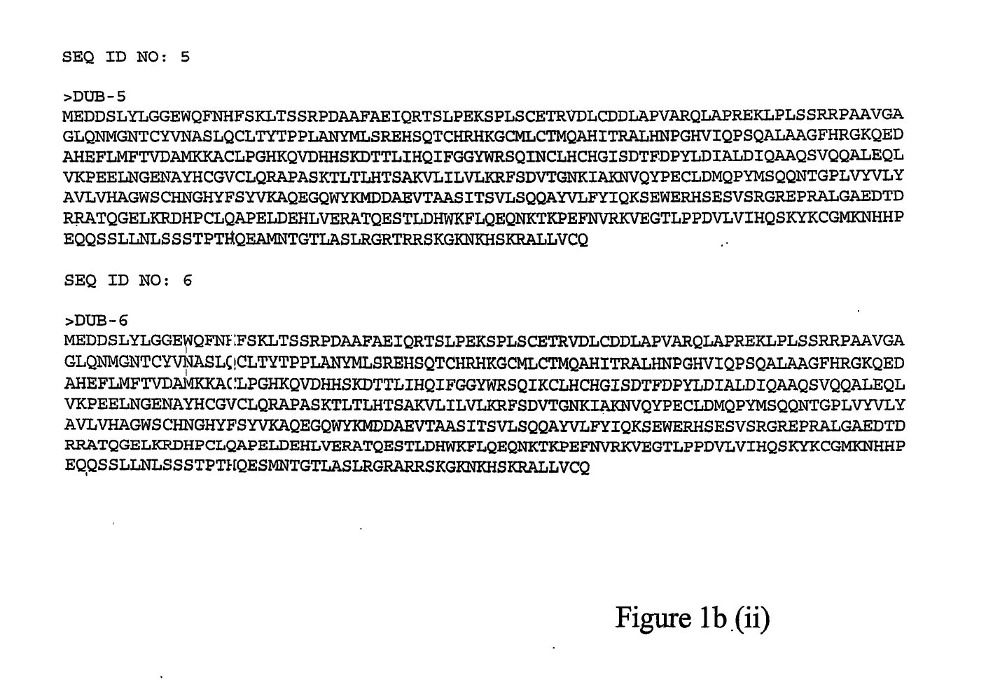

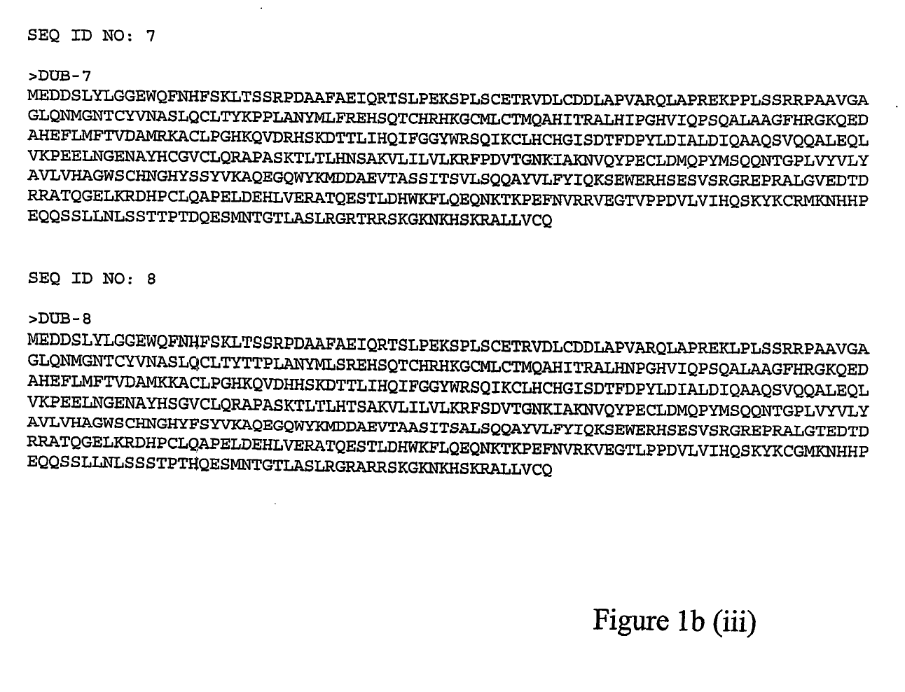

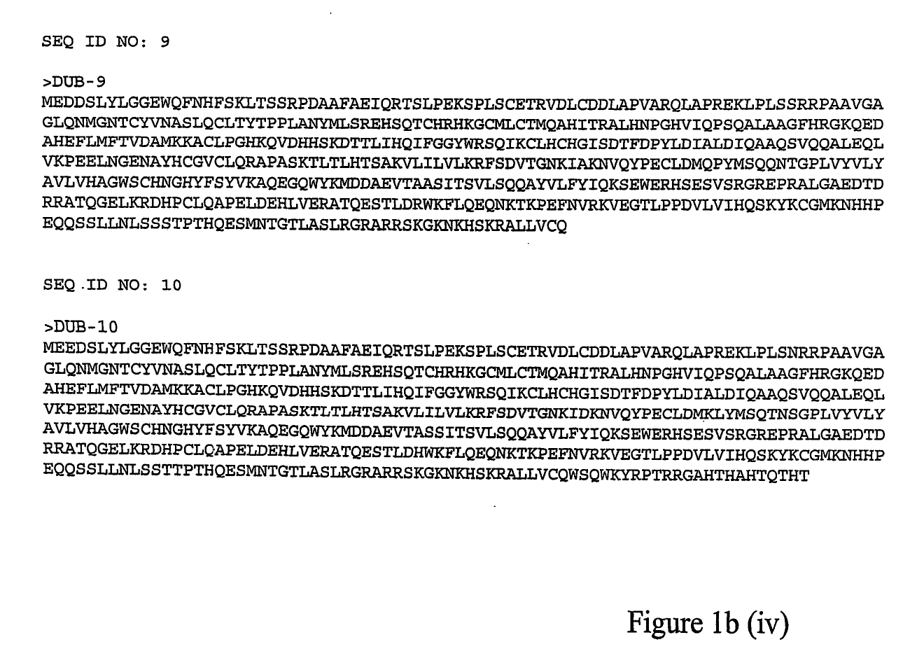

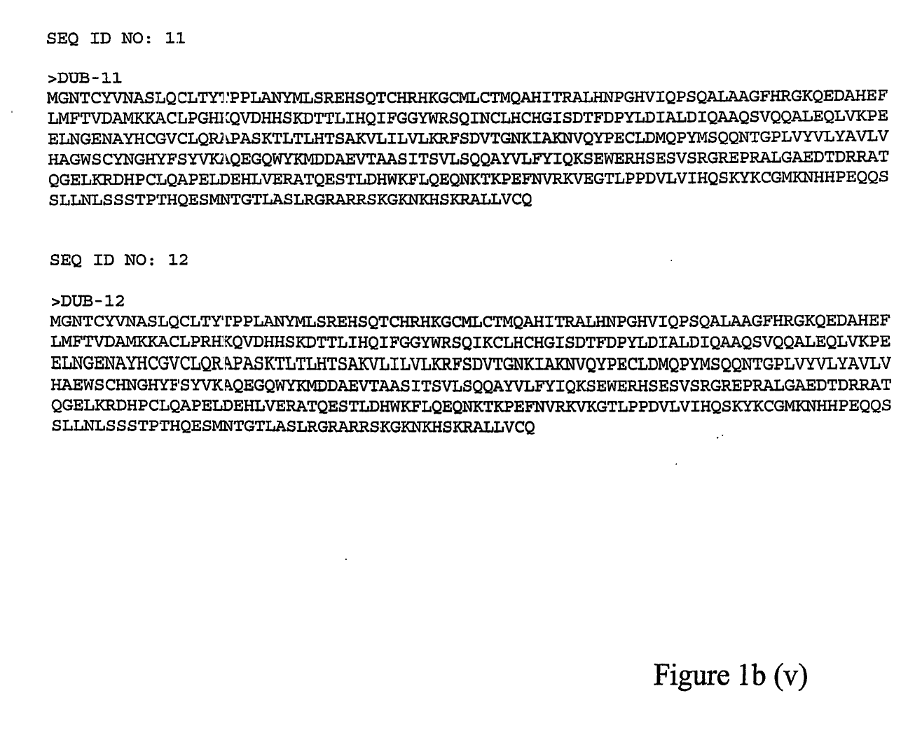

Accordingly, in a first aspect of the present invention, there is provided an isolated polypeptide comprising the amino acid sequence shown as DUB-3 in FIG. 1 (SEQ ID NO: 1), or a variant or fragment thereof.

In preferred embodiments of the invention, the polypeptide comprises the amino acid sequence shown as DUB-3 in FIG. 1 (SEQ ID No: 1). In further preferred embodiments of the invention, the polypeptide consists of the acid sequence shown as DUB-3 in FIG. 1 (SEQ ID No: 1).

In preferred embodiments of the invention a variant or fragment of SEQ ID NO: 1 shows greater than 90% homology, preferably more than 95% homology , even more preferably greater than 97%, yet more preferably greater than 98% homology, most preferably greater than 99% homology with the sequence of SEQ ID NO: 1. Preferably, variants do not include the RS447 sequence (Accession no D38378) (Genomics Vol 67 p291 (2000)).

In preferred embodiments, the isolated polypeptide is a human polypeptide.

Also encompassed by the first aspect of the invention there is provided an isolated polypeptide comprising the amino acid sequence shown as DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11 (SEQ ID NO: 11) or DUB-12 (SEQ ID NO: 12) in FIG. 1 , or a variant or fragment thereof.

In preferred embodiments of the invention a variant or fragment of any one of SEQ ID NO: 4 to SEQ ID NO: 12, shows greater than 90% homology, preferably more than 95% homology , even more preferably greater than 97%, yet more preferably greater than 98% homology, most preferably greater than 99% homology with the sequence of the corresponding polypeptide (i.e. of SEQ ID NO: 4 to SEQ ID NO: 12). Preferably, variants do not include the RS447 sequence (Accession no D38378) (Genomics Vol 67 p291 (2000)).

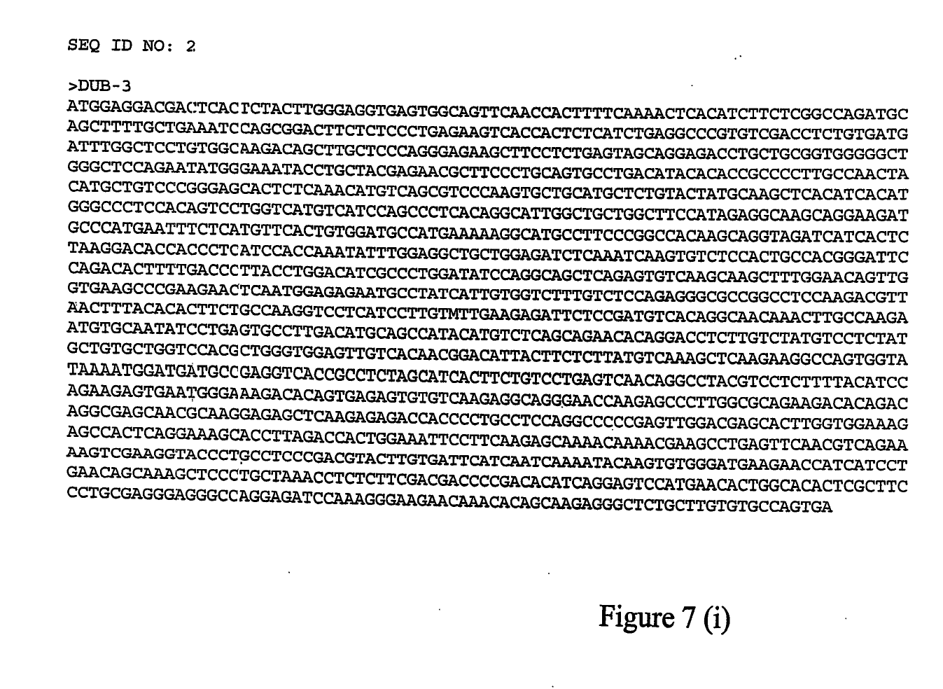

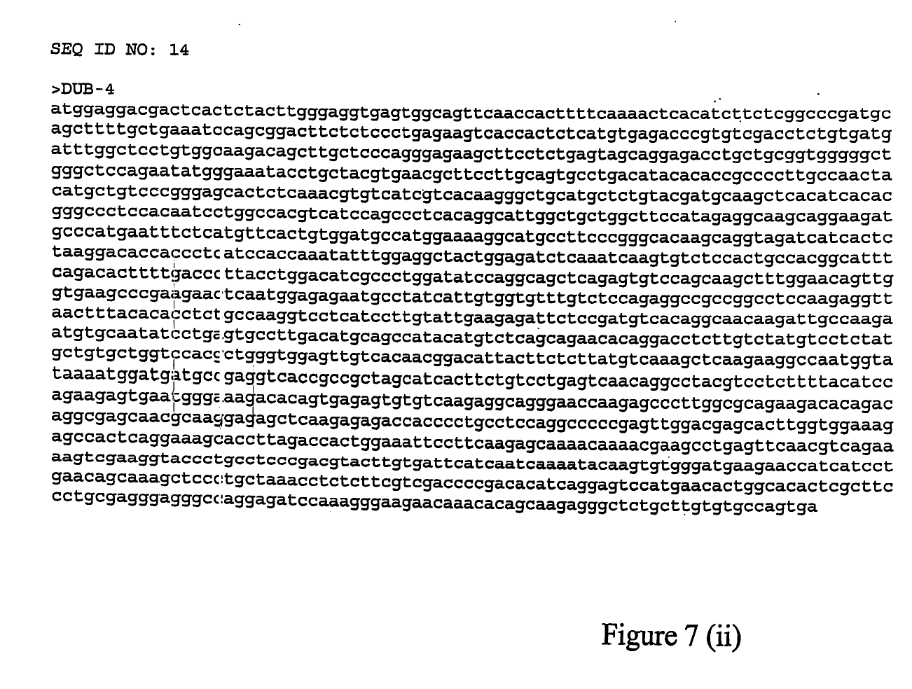

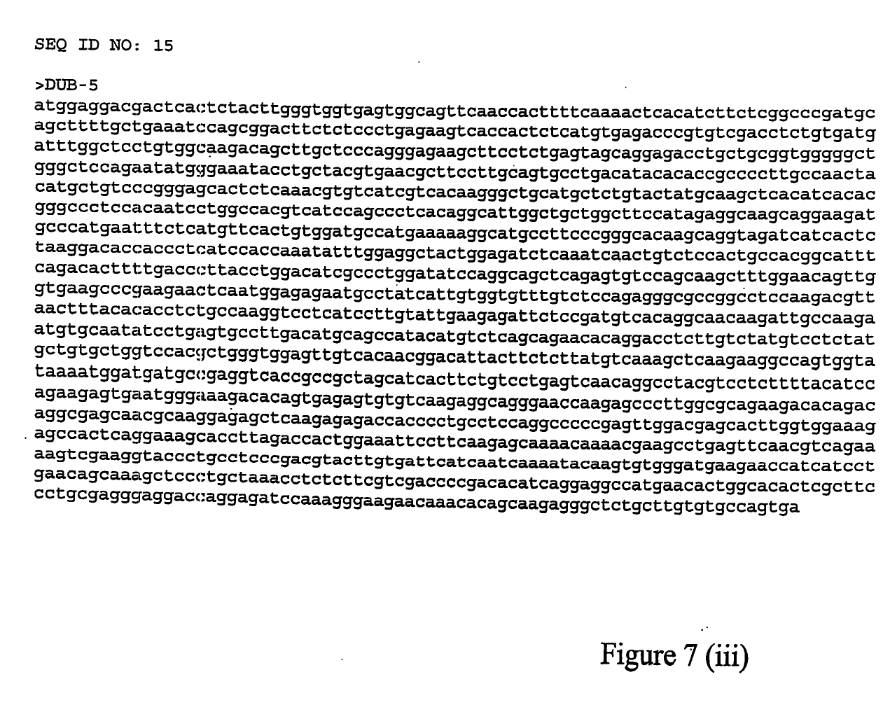

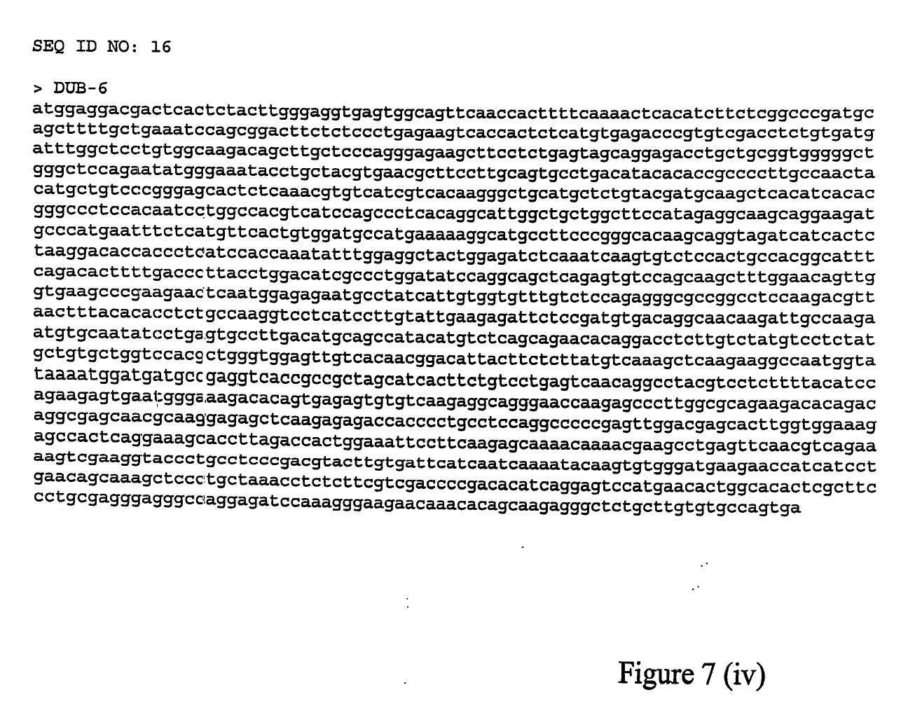

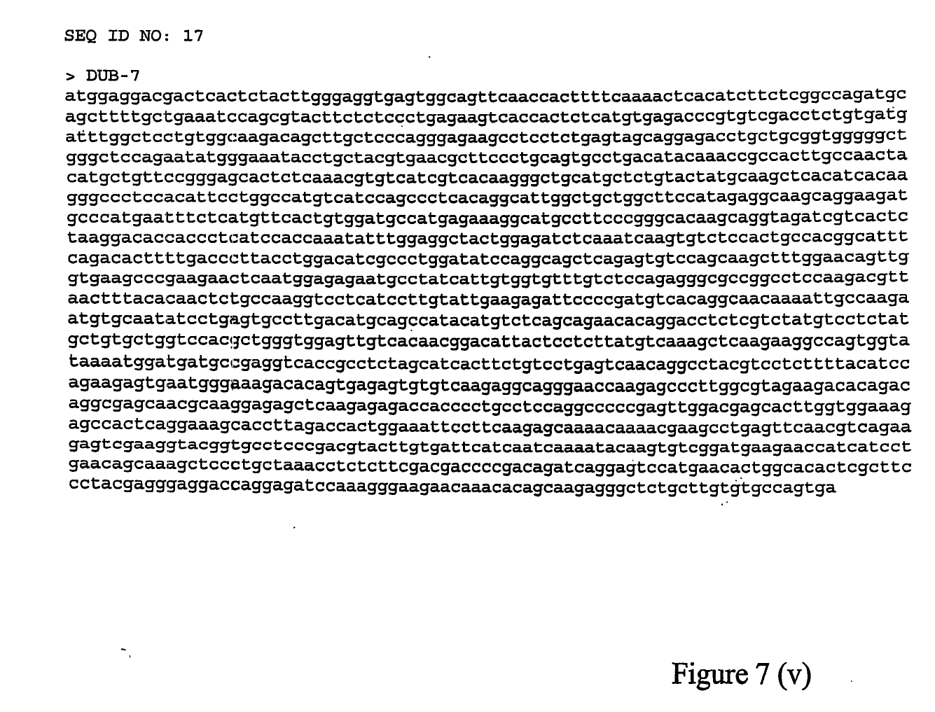

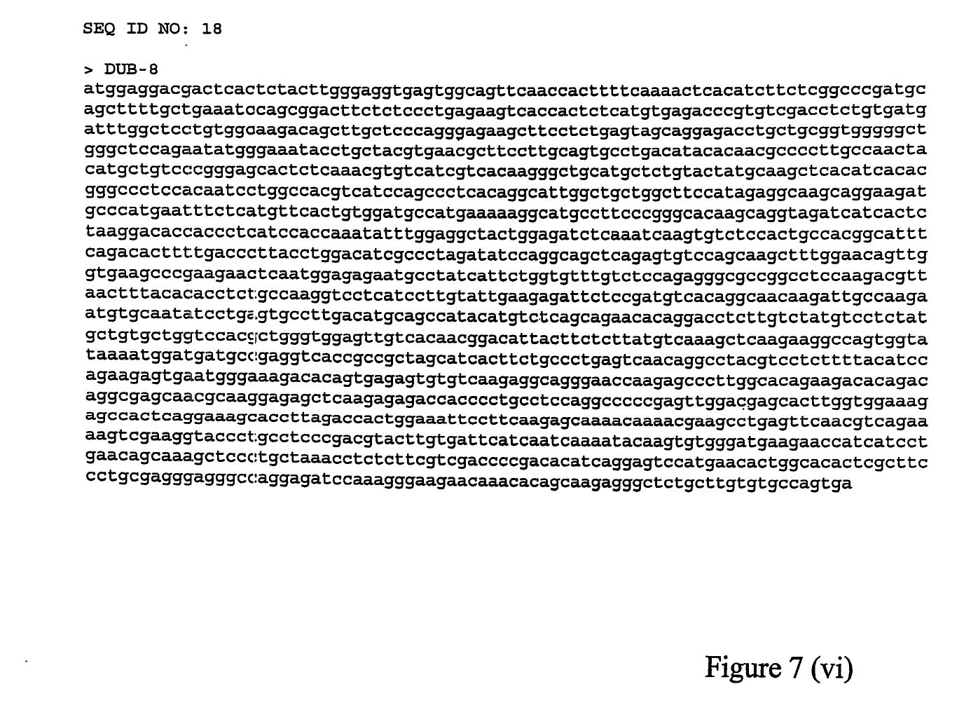

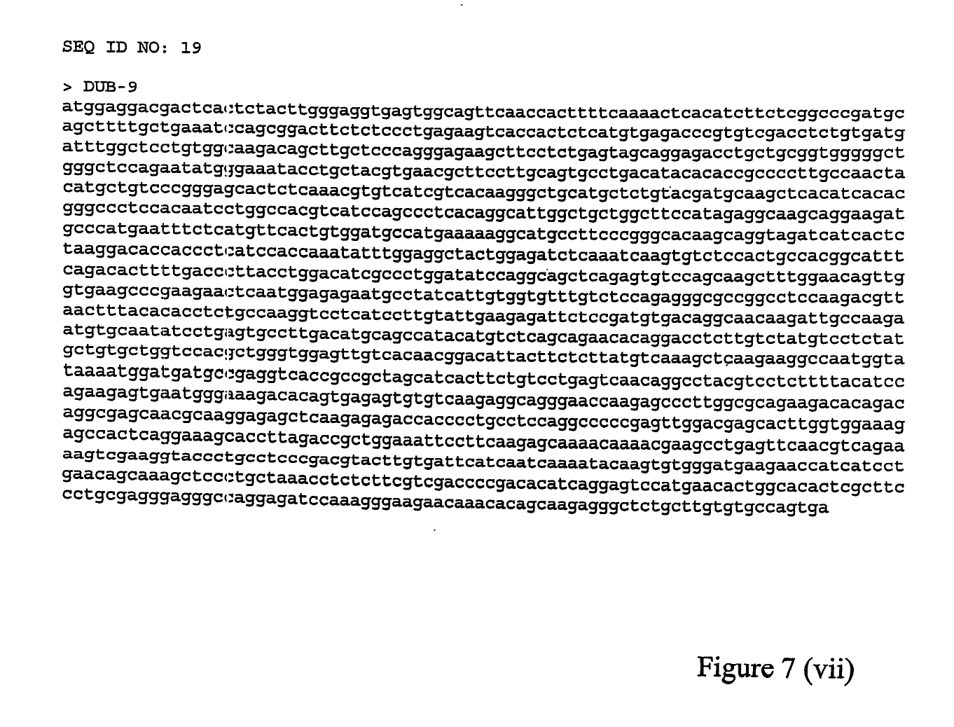

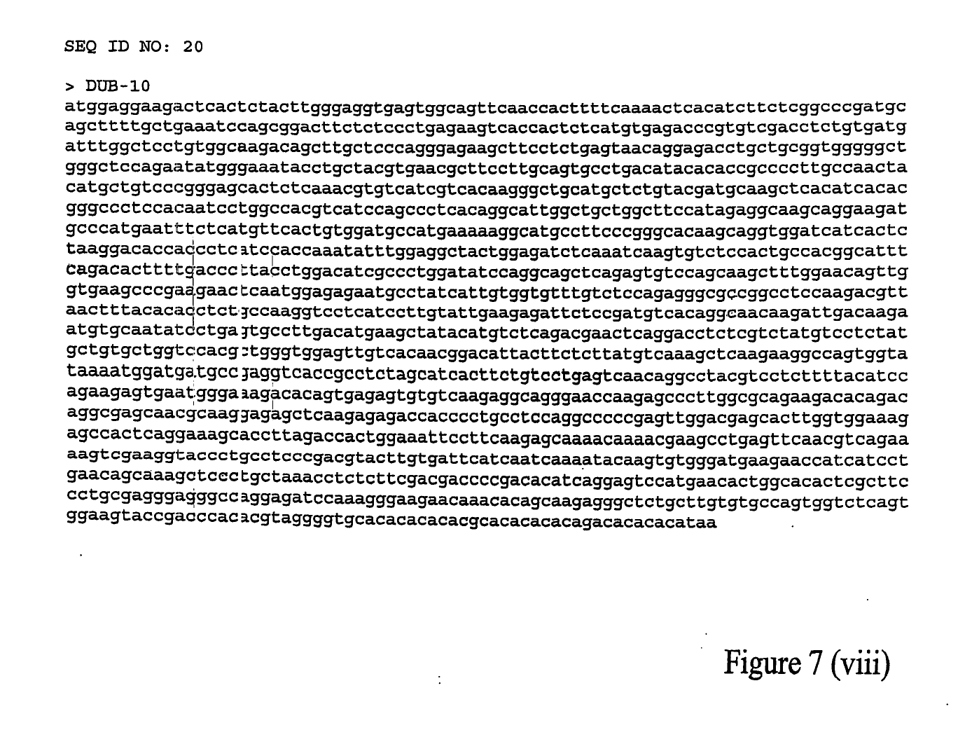

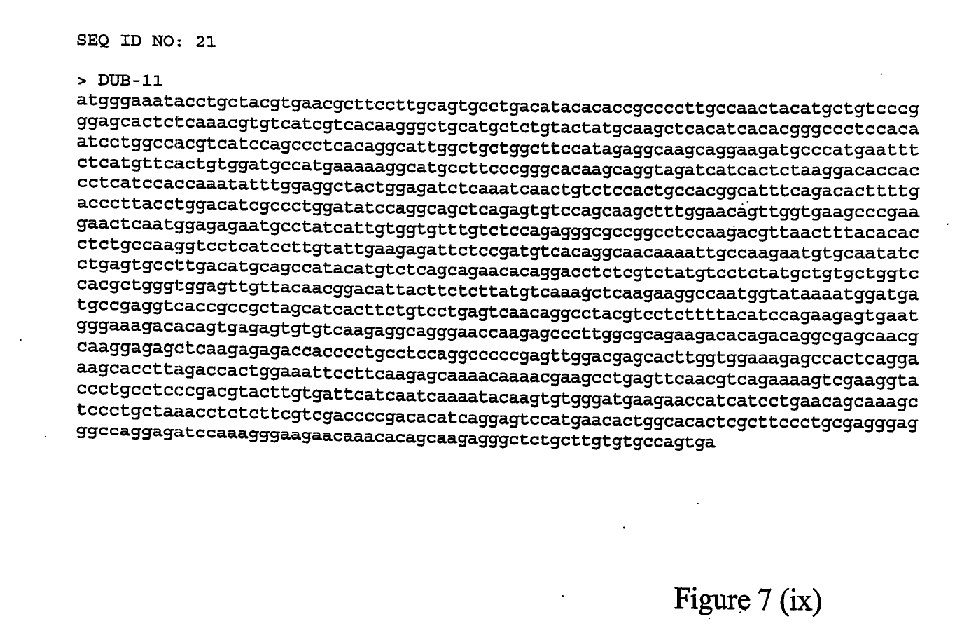

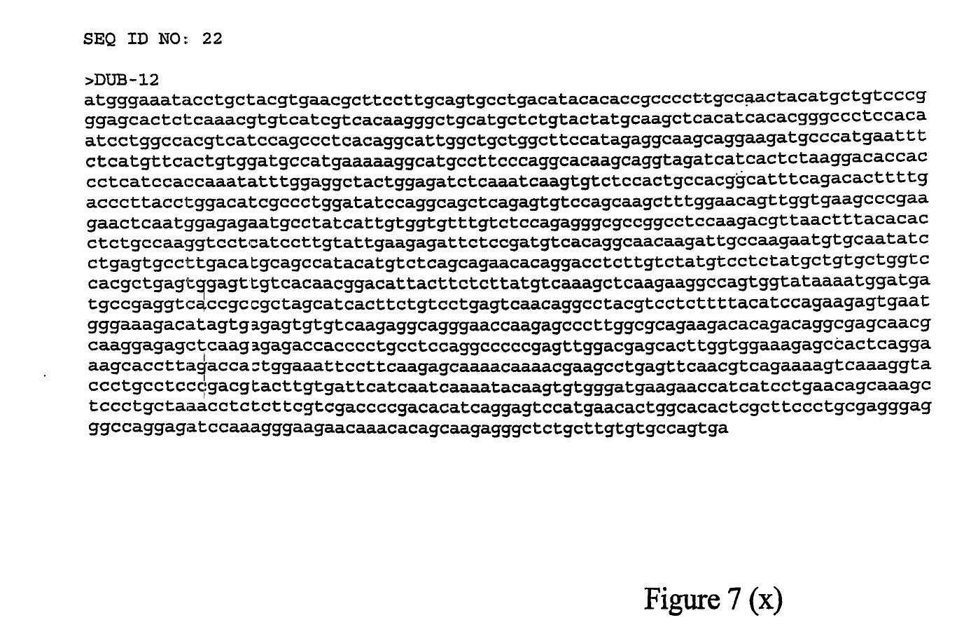

In a second aspect, there is provided an isolated nucleic acid sequence encoding a polypeptide which includes the amino acid sequence shown as DUB-3 in FIG. 1 (SEQ ID NO: 1) or a variant or fragment thereof. In a preferred aspect of the invention, the nucleic acid molecule comprises the nucleotide sequence shown in FIG. 7 (SEQ ID NO: 2)or a variant or fragment thereof. In a further preferred embodiment, the nucleic acid molecule consists of the nucleotide sequence shown in FIG. 7 (SEQ ID NO: 2).

In preferred embodiments of the invention a variant or fragment of SEQ ID NO: 2 shows greater than 90% homology, preferably more than 95% homology , even more preferably greater than 97%, yet more preferably greater than 98% homology, most preferably greater than 99% homology with the sequence of SEQ ID NO: 2. Preferably, variants do not include the nucleic acid sequence encoding the RS447 sequence (Accession no D38378) (Genomics Vol 67 p291 (2000)).

Also encompassed by the second aspect, there is provided an isolated nucleic acid sequence encoding a polypeptide which includes the amino acid sequence shown as DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11 (SEQ ID NO: 11) or DUB-12 (SEQ ID NO: 12) of FIG. 1 or a variant or fragment thereof. In preferred aspects of the invention, the nucleic acid molecule comprises one of the nucleotide sequences shown for DUB-4 (SEQ ID NO: 14), DUB-5 (SEQ ID NO: 15), DUB-6 (SEQ ID NO: 16), DUB-7 (SEQ ID NO: 17), DUB-8 (SEQ ID NO: 18), DUB-9 (SEQ ID NO: 19), DUB-10 (SEQ ID NO: 20), DUB-11 (SEQ ID NO: 21) or DUB-12 (SEQ ID NO: 22) in FIG. 7 or a variant or fragment thereof.

In a further preferred embodiment, the nucleic acid molecule consists of the nucleotide sequence shown in FIG. 7 as DUB-4 (SEQ ID NO: 14), DUB-5 (SEQ ID NO: 15), DUB-6 (SEQ ID NO: 16), DUB-7 (SEQ ID NO: 17), DUB-8 (SEQ ID NO: 18), DUB-9 (SEQ ID NO: 19), DUB-10 (SEQ ID NO: 20), DUB-11 (SEQ ID NO: 21) or DUB-12 (SEQ ID NO: 22).

In preferred embodiments of the invention a variant or fragment of any one of SEQ ID NO: 14 to SEQ ID NO: 22 shows greater than 90% homology, preferably more than 95% homology , even more preferably greater than 97%, yet more preferably greater than 98% homology, most preferably greater than 99% homology with the sequence of the corresponding polypeptide (i.e. of SEQ ID NO: 14 to SEQ ID NO: 22). Preferably, variants do not include the nucleic acid sequence encoding the RS447 sequence (Accession no D38378) (Genomics Vol 67 p291 (2000)).

Also provided by the invention in a third aspect is a specific binding member, for example an antibody, which binds to a polypeptide of the invention.

Such specific binding members may be used for a variety of purposes, including assays and diagnostic methods to identify the presence of the polypeptides of the invention in a sample.

Accordingly, in a further aspect of the invention, there is provided a method for identifying the presence of a polypeptide according to the first aspect of the invention in a biological sample, said method including the steps:
a) bringing said biological sample into contact with a specific binding member of the third aspect of the invention;
b) determining binding of said specific binding member to said sample, wherein binding of said specific binding member is indicative of the presence of said polypeptide in said sample.

As described herein, DUB-3 has been found to be differentially expressed in normal and tumour cells of some tissues. Thus the polypeptides and nucleic acids of the invention may be used in assays to determine susceptibility to cancer of particular tissues or indeed to diagnose cancer in tissues.

Accordingly, in a fifth aspect of the invention, there is provided a method of determining susceptibility to cancer in a patient, said method comprising the steps:
a) providing a biological sample from a patient,
b) bringing said biological sample into contact with a specific binding member of the third aspect of the invention, and
c) detecting binding of the specific binding member to said sample, wherein binding of said specific binding member is indicative of susceptibility to cancer.

In preferred aspects of the invention, the cancer is a haematopoietic cancer, lung cancer, skin cancer, small intestinal cancer or thymus cancer.

In particularly preferred embodiments of the invention, the cancer is promyelocytic leukaemia, chronic myelogenous leukaemia, lymphoblastic leukaemia, Burkitt's lymphoma, cancer of the lung or cancer of the spleen.

The invention may also be used to diagnose and/or monitor progression of cancer in a patient.

Thus, in a sixth aspect, there is provided a method of diagnosis of cancer in a patient, said method comprising the steps:
a) providing a biological sample from a patient,
b) bringing said biological sample into contact with a specific binding member of the third aspect of the invention, and
c) detecting binding of the specific binding member to said sample, wherein binding of said specific binding member is indicative of cancer.

In alternative embodiments, expression of the DUB-3 gene (or indeed any one of DUB-4 to DUB-12 genes) may be assayed to determine susceptibility.

Accordingly, in a seventh aspect of the invention, there is provided a method of determining susceptibility to cancer in a patient, said method comprising the steps:
a) providing a biological sample from said sample,
b) determining the presence of a DUB-3, DUB-4, DUB-5, DUB-6, DUB-7, DUB-8, DUB-9, DUB-10, DUB-11 or DUB-12 nucleic acid or a fragment thereof in said sample, wherein the presence of said nucleic acid or fragment thereof is indicative of a predisposition to cancer.

In a preferred embodiment, the method comprises the steps:
a) providing a biological sample from a patient,
b) bringing said biological sample into contact with a nucleic acid molecule which is capable of hybridising, under stringent conditions, to a nucleic acid molecule according to the second aspect of the invention, under conditions which allow hybridisation of substantially complementary nucleic acids, and
c) detecting hybridisation of nucleic acids.

In an eighth aspect, there is provided a method of diagnosis of cancer, said method comprising the steps:
a) providing a biological sample from said sample,
b) determining the presence or relative amount of a DUB-3, DUB-4, DUB-5, DUB-6, DUB-7, DUB-8, DUB-9, DUB-10, DUB-11 or DUB-12 nucleic acid or fragment thereof in said sample, wherein the presence of or presence above a predetermined minimum of said nucleic acid or fragment thereof is indicative of cancer.

In one embodiment the predetermined minimum is an expression level of the nucleic acid or fragment in a control sample known to be free of cancerous cells.

In a preferred embodiment, said method comprises the steps:
a) providing a biological sample from a patient,
b) bringing said biological sample into contact with a nucleic acid molecule which is capable of hybridising, under stringent conditions, to a nucleic acid molecule according to the second aspect of the invention, under conditions which allow hybridisation of substantially complementary nucleic acids, and
c) detecting hybridisation of nucleic acids, wherein detection of hybridisation is indicative of the presence of cancer.

As described further in the Examples, the inventors have shown that constitutive expression of DUB-3 blocks proliferation and leads to an increase in the number of apoptotic cells observed. This clearly suggests that modulation of DUB-3 expression (or indeed of expression of any one of DUB-4 to DUB-12) may be employed in methods of treatment of cancers.

Therefore, a ninth aspect of the present invention provides a method of treatment of cancer in a patient in need thereof, said method comprising the step of administering an agent which modulates the expression of any one of DUB-3 to DUB-12, preferably DUB-3, in the cancer cells.

In one embodiment, the agent, which may be peptide or non-peptide, enhances expression of any one of DUB-3 to DUB-12, preferably DUB-3.

However, given the enhanced expression of DUB-3 in many cancer cell lines and given the observed tight regulation of DUB-3 expression, the inventors believe that, in many cases, for example, in growth factor deprived tumour cells, excessive expression of DUB-3 (or indeed of any one of DUB-3 to DUB-12), may in fact stimulate tumour growth.

Thus, in further embodiments of the ninth aspect of the invention, the agent inhibits expression of any one of DUB-3 to DUB-12, preferably DUB-3. Suitable agents may be peptide or non-peptide. In one embodiment, the agent is a specific binding member according to the third aspect of the invention. In a further embodiment, the agent for use in the ninth aspect of the invention may be a nucleic acid molecule, for example an antisense polynucleotide, wherein the polynucleotide is antisense to the nucleic acid of the first aspect of the invention.

Moreover, the invention may be used to identify agents useful in the methods of the invention. Accordingly, in a tenth aspect of the present invention, there is provided an assay method for identifying an agent having anti-proliferative activity, said method comprising the steps of:
a) bringing a candidate agent into contact with a polypeptide according to the first aspect of the invention
c) determining interaction or binding between the candidate agent and the polypeptide.

Novel agents identified by such a method of the invention fall within the scope of the present invention. The agents may be formulated into a medicament.

The invention further provides the use of a polypeptide according to the first aspect of the invention, a nucleic acid according to the second aspect of the invention or an agent which modulates expression of the polypeptide according to the first aspect of the invention in the preparation of a medicament for the treatment of cancer.

Also provided is a polypeptide according to the first aspect of the invention, a nucleic acid according to the second aspect of the invention or a novel agent which modulates expression of the polypeptide according to the first aspect of the invention for use in medicine, preferably for use in the treatment of cancer.

As described in the Examples, the inventors have shown that DUB-3 expression down regulates the RAS/MEK/ERK signalling pathway. They have shown that in the presence of DUB-3 expression, a slower migrating form of Ras is observed. Furthermore, the inventors have shown that RCE1 is a DUB-3 substrate.

Without being limited by any one theory, the inventors believe that DUB-3 blocks the degradation of Ras converting enzyme 1 (RCE1). RCE1 is involved in the processing of multiple CAAX motif (where C=Cys, A=aliphatic amino acid, X=any amino acid). containing proteins in addition to Ras (for example Rho, Rap, CDC42) and it can therefore be surmised that DUB-3 expression could influence their activation through its affects on RCE1.

Accordingly, in a further aspect of the invention, there is provided a method of modulating RCE1 levels in a cell, said method comprising the step of administering an agent which modulates the expression of any one of DUB-3 to DUB-12, preferably DUB-3, in the cells.

In another aspect, there is provided a method of modulating the concentration of CAAX motif containing proteins in a cell, said method comprising the step of administering an agent which modulates the expression of any one of DUB-3 to DUB-12, preferably DUB-3, in the cells.

In one embodiment, the CAAX motif containing protein is Ras. In other embodiments, the CAAX motif containing protein is Rho, Rap or CDC42.

Enhanced expression of such CAAX motif containing proteins are believed to be tumourigenic.

Accordingly, the use of DUB-3 modulators, in particular agents which increase DUB-3 may be advantageous in decreasing activation of such CAAX motif containing proteins and thus may be used in the treatment of cancers.

Accordingly, in a further aspect, there is provided a method of treatment of cancer in a patient in need thereof, said method comprising the step of administering an agent which increases the expression of any one of DUB-3 to DUB-12, preferably DUB-3, in the cancer cells. In a preferred embodiment, the cancer is a cancer associated with increased expression of a CAAX motif containing proteins.

Also provided is the use of an agent which increases the expression of any one of DUB-3 to DUB-12 in the preparation of a medicament for the treatment of a cancer associates with increased expression of a CAAX motif containing protein.

Preferred features of each aspect of the invention are as for each of the other aspects mutatis mutandis unless the context demands otherwise

### DETAILED DESCRIPTION

Polypeptides

The terms peptide, polypeptide and protein are used interchangeably within this specification.

As described herein, the inventors have found a novel deubiquitinating enzyme, which is differentially expressed in tumour cells. The polypeptide of the invention has been named DUB-3. The sequence is shown as SEQ ID NO: 1 of FIG. 1.

However, unless the context demands otherwise, a variant or fragment of any one of DUB-3, DUB-4, DUB-5, DUB-6, DUB-7, DUB-8, DUB-9, DUB-10, DUB-11 or DUB-12 does not include the RS447 sequence (Accession no D38378) (Genomics Vol 67 p291 (2000)). Moreover, the such variants or fragments do not include the DUB-1 or DUB-2 sequences shown in FIG. 1.

In preferred embodiments of the invention, the DUB-3 polypeptide of the invention consists of the polypeptide having the amino acid sequence shown as SEQ ID NO: 1.

Preferred variants of SEQ ID NO: 1 show greater than 90% homology, preferably more than 95% homology, even more preferably greater than 97%, yet more preferably greater than 98% homology, most preferably greater than 99% homology with the sequence of SEQ ID NO: 1. Homology comparisons may be conducted by eye, or more usually with the aid of readily available sequence comparison programs.

The percent identity of two amino acid sequences or of two nucleic acid sequences may be determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions=100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilised for the comparison of sequences is the algorithm of Myers & Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis & Robotti (1994) Comput. Appl. Biosci., 10 :3-5; and FASTA described in Pearson & Lipman (1988) Proc. Natl. Acad. Sci. 85:2444-8. Within FASTA, ktup is a control option that sets the sensitivity and speed of the search.

Where high degrees of sequence identity are present there will be relatively few differences in amino acid sequence. Thus for example there may be less than 40, for example less than 30, less than 20, less than 10, or even less than 5 differences.

A fragment of a DUB polypeptide in accordance with the invention can be any length up to the full length of the relevant DUB-polypeptide (DUB-3, DUB-4, DUB-5, DUB-6, DUB-7, DUB-8, DUB-9, DUB-10, DUB-11 or DUB-12). For example, a fragment of a DUB-3 polypeptide in accordance with the invention can be any length up to the full length of the DUB-3 polypeptide shown in SEQ ID NO: 1; it thus encompasses DUB-3 polypeptides which have been truncated by a few amino acids, for example, less than 20 amino acids, as well as shorter fragments. A fragment of a polypeptide generally means a stretch of amino acids of at least 5, typically at least 10, preferably at least 20 contiguous amino acids. Most preferably a fragment of the polypeptide of the invention has at least 40 or more, for example, 50, 60, 70, 80, 90 or 100 contiguous amino acids. In one preferred embodiment, fragments according to the invention are between about 10 and 50 amino acids.

Similarly, a fragments of any one of DUB4 to DUB 12 may be any length up to the full length of the relevant DUB polypeptide

Fragments of the invention may be useful in raising antibodies to a portion of the relevant DUB sequence. Accordingly, in preferred embodiments of the invention, a fragment of the invention includes at least one antigenic determinant (epitope) characteristic of DUB-3 (or indeed of any one of DUB-4 to DUB-12). Whether or not a particular polypeptide fragment retains such antigenic properties can readily be determined by routine methods in the art.

Nucleic Acid

A “nucleic acid” of the present invention is a nucleic acid which encodes a DUB-3 polypeptide (or indeed a DUB-4, DUB-5, DUB-6, DUB-7, DUB-8, DUB-9, DUB-10, DUB-11 or DUB-12 polypeptide), as described above, preferably a human DUB-3 polypeptide. The term moreover includes polynucleotides capable of hybridising, under stringent hybridisation conditions, to the naturally occurring nucleic acids identified above, or the complement thereof.

“Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5× Denhardt's solution, 10% dextran sulphate, and 20 pg/ml denatured, sheared salmon sperm DNA, followed by washing the filters in 0.1×SSC at about 65° C.

A nucleic acid encoding a fragment according to the invention can be the result of nucleic acid amplification of a specific region of a DUB-3 gene (or indeed a specific region of a DUB-4, DUB-5, DUB-6, DUB-7, DUB-8, DUB-9, DUB-10, DUB-11 or DUB-12 gene), incorporating a mutation in accordance with the present invention.

Although nucleic acids, as referred to herein, are generally natural nucleic acids found in nature, the term can include within its scope modified, artificial nucleic acids having modified backbones or bases, as are known in the art.

Nucleic acid for use in accordance with the present invention may comprise DNA or RNA and may be wholly or partially synthetic. In a preferred aspect, nucleic acid for use in the invention codes for a binding member of the invention as defined above. The skilled person will be able to determine substitutions, deletions and/or additions to such nucleic acids which will still provide a binding member suitable for use in the present invention.

Nucleic acid sequences encoding a peptide in accordance with the present invention can be readily prepared by the skilled person using the information and references contained herein and techniques known in the art. (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992). These techniques include (i) the use of the polymerase chain reaction (PCR) to amplify samples of such nucleic acid, e.g. from genomic sources; (ii) chemical synthesis, or (iii) preparing cDNA sequences. DNA encoding polypeptides may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

Modifications to the sequences can be made, e. g. using site directed mutagenesis, to lead to the expression of modified peptide or to take account of codon preference in the host cells used to express the nucleic acid.

In order to obtain expression of the nucleic acid sequences, the sequences can be incorporated in a vector having one or more control sequences operably linked to the nucleic acid to control its expression. The vectors may include other sequences such as promoters or enhancers to drive the expression of the inserted nucleic acid, nucleic acid sequences so that the peptide is produced as a fusion and/or nucleic acid encoding secretion signals so that the polypeptide produced in the host cell is secreted from the cell.

Peptides can then be obtained by transforming the vectors into host cells in which the vector is functional, culturing the host cells so that the peptide is produced and recovering the peptide from the host cells or the surrounding medium.

Thus, the present invention also encompasses a method of making a peptide of the first aspect of the invention, the method including expression from nucleic acid encoding the peptide (generally nucleic acid according to the second aspect of the invention). This may be achieved by growing a host cell in culture, containing such a vector; under appropriate conditions which cause or allow expression of the polypeptide.

Suitable vectors can be chosen or constructed, containing appropriate regulatory sequences, including promoter sequences, terminator fragments, polyadenylation sequences, enhancer sequences, marker genes and other sequences as appropriate.

Vectors may be plasmids, viral e. g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Systems for cloning and expression of a polypeptide in a variety of different host cells are well known. Suitable host cells include bacteria, eukaryotic cells such as mammalian and yeast, and baculovirus systems.

Thus, a further aspect of the present invention provides a host cell containing-heterologous nucleic acid as disclosed herein.

The nucleic acid of the invention may be integrated into the genome (e. g. chromosome) of the host cell.

Integration may be promoted by inclusion of sequences which promote recombination with the genome, in accordance with standard techniques. The nucleic acid may be on an extra-chromosomal vector within the cell, or otherwise identifiably heterologous or foreign to the cell.

The introduction, which may (particularly for in vitro introduction) be generally referred to without limitation as “transformation”, may employ any available technique.

For eukaryotic cells suitable techniques may include calcium phosphate transfection, DEAE-Dextran, electroporation, liposome-mediated transfection and transduction using retrovirus or other virus, e. g. vaccinia or, for insect cells, baculovirus. For bacterial cells, suitable techniques may include calcium chloride transformation, electroporation and transfection using bacteriophage. As an alternative, direct injection of the nucleic acid could be employed.

The introduction may be followed by causing or allowing expression from the nucleic acid, e. g. by culturing host cells (which may include cells actually transformed although more likely the cells will be descendants of the transformed cells) under conditions for expression of the gene, so that the encoded polypeptide (or peptide) is produced. If the polypeptide is expressed coupled to an appropriate signal leader peptide it may be secreted from the cell into the culture medium. Following production by expression, a polypeptide or peptide may be isolated and/or purified from the host cell and/or culture medium, as the case may be, and subsequently used as desired, e. g. in the formulation of a composition which may include one or more additional components, such as a pharmaceutical composition which includes one or more pharmaceutically acceptable excipients, vehicles or carriers (e. g. see below).

Nucleic acid molecules and vectors for use in accordance with the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The nucleic acid may be integrated into the genome (e.g. chromosome) of the host cell. Integration may be promoted by inclusion of sequences which promote recombination with the genome in accordance with standard techniques. The nucleic acid may be on an extra-chromosomal vector within the cell, or otherwise identifiably heterologous or foreign to the cell.

The present invention further extends to methods of gene therapy-using the nucleic acid molecules of the present invention

Binding Members

In the context of the present invention, a “binding member” is a molecule which has binding specificity for another molecule, in particular a DUB-3 (or indeed a DUB-4, DUB-5, DUB-6, DUB-7, DUB-8, DUB-9, DUB-10, DUB-11 or DUB-12) polypeptide or fragment thereof. The binding member may be a member of a pair of specific binding members. The members of a binding pair may be naturally derived or wholly or partially synthetically produced. A binding member of the invention and for use in the invention may be any moiety, for example an antibody or ligand, which can specifically bind to a DUB-3 polypeptide (or a DUB-4, DUB-5, DUB-6, DUB-7, DUB-8, DUB-9, DUB-10, DUB-11 or DUB-12 polypeptide).

Antibodies

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd; and diabodies.

The binding member of the invention may be an antibody such as a monoclonal or polyclonal antibody, or a fragment thereof. The constant region of the antibody may be of any class including, but not limited to, human classes IgG, IgA, IgM, IgD and IgE. The antibody may belong to any sub class e.g. IgG1, IgG2, IgG3 and IgG4. IgG1 is preferred.

As antibodies can be modified in a number of ways, the term “antibody” should be construed as covering any binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of such binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (W094/13804; P. Hollinger et al., Proc. Natl. Acad. Sci. USA 90:6444-6448 (1993)).

A fragment of an antibody or of a polypeptide for use in the present invention generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having DUB activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody and the constant region of a human antibody. Thus the binding member may comprise a human constant region.

The variable region other than the hypervariable region may also be derived from the variable region of a human antibody and/or may also be derived from a monoclonal antibody. In such case, the entire variable region may be derived from murine monoclonal antibody and the antibody is said to be chimerised. Methods for making chimerised antibodies are known in the art. Such methods include, for example, those described in U.S. patents by Boss (Celltech) and by Cabilly (Genentech). See U.S. Pat. Nos. 4,816,397 and 4,816,567, respectively.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRS), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

Production of Binding Members

The binding members for use in the present invention may be generated naturally, or wholly or partly by chemical synthesis. The binding members can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a binding member suitable for use in the present invention is to express nucleic acid encoding it, by use of nucleic acid in an expression system. Thus the present invention further provides the use of (a) nucleic acid encoding a specific binding member which binds to a polypeptide of the invention and, optionally, (b) a chemotherapeutic agent in the preparation of a medicament for treating cancer.

The expression of antibodies and antibody fragments in prokaryotic cells such as E. coli is well established in the art. For a review, see for example Plückthun, Bio/Technology 9:545-551 (1991). Expression in eukaryotic cells in culture is also available to those skilled in the art as an option for production of a binding member, see for recent review, for example Reff, Curr. Opinion Biotech. 4:573-576 (1993); Trill et al., Curr. Opinion Biotech. 6:553-560 (1995).

Pharmaceutical Compositions

As described above, the present invention extends to a pharmaceutical composition for the treatment of cancer, the composition comprising a) a nucleic acid, polypeptide, binding member and/or composition of the invention and b) a pharmaceutically acceptable excipient, diluent or carrier.

Pharmaceutical compositions according to the present invention, and for use in accordance with the present invention, may comprise, in addition to active ingredients, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

Administration

Nucleic acids, polypeptides, binding members and/or compositions of the invention may be administered in any suitable way. Moreover each of them may be used in combination therapy with other treatments, for example, other chemotherapeutic agents. In such embodiments, the nucleic acids, polypeptides, binding members and/or compositions of the invention may be administered simultaneously, separately or sequentially with another chemotherapeutic agent.

Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

In a preferred embodiment, the nucleic acids, polypeptides, binding members and/or compositions of the invention are administered as a pharmaceutical composition, which will generally comprise a suitable pharmaceutical excipient, diluent or carrier selected dependent on the intended route of administration.

Nucleic acids, polypeptides, binding members and/or compositions (agents) of and for use in the present invention may be administered to a patient in need of treatment via any suitable route.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The nucleic acids, polypeptides, binding members and/or compositions of the invention may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218,121A; Epstein et al, PNAS USA, 82: 3688-3692, 1985; Hwang et al, PNAS USA, 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

The nucleic acids, polypeptides, binding members, agent, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Dose

The nucleic acid, polypeptide, binding member and/or composition of the invention are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

Therapeutic Uses

The peptides and methods of the invention may be used in the treatment of any cancer.

“Treatment” or “therapy” includes any regime that can benefit a human or non-human animal. The treatment may be in respect of an existing condition or may be prophylactic (preventative treatment). Treatment may include curative, alleviation or prophylactic effects.

The nucleic acids, polypeptides, specific binding members, compositions and methods of the invention may be particularly useful in the treatment of existing cancer and in the prevention of the recurrence of cancer after initial treatment or surgery.

In a preferred embodiment, the nucleic acids, polypeptides, specific binding members, compositions and methods are used for the treatment of haematopoietic cancer, lung cancer, skin cancer, small intestinal cancer or thymus cancer.

Assays

In various further aspects the present invention relates to screening and assay methods and means, and substances identified thereby.

Thus, further aspects of the present invention provide the use of a polypeptide or peptide particularly a fragment of a polypeptide of the invention as disclosed, and/or encoding nucleic acid therefor, in screening or searching for and/or obtaining/identifying a substance, e.g. peptide or chemical compound, which interacts and/or binds with the polypeptide or peptide and/or interferes with its function or activity or that of another substance, e.g. polypeptide or peptide, which interacts and/or binds with the polypeptide or peptide of the invention.

For instance, a method according to one aspect of the invention a polypeptide or peptide of the invention and bringing it into contact with a substance, which contact may result in binding between the polypeptide or peptide and the substance. Binding may be determined by any of a number of techniques available in the art, both qualitative and quantitative.

Further assays are for substances which interact with or bind a polypeptide of the invention and/or modulate one of more of its activities.

One aspect of the present invention provides an assay which includes:
(a) bringing into contact a polypeptide or peptide according to the invention and a putative binding molecule or other test substance; and
(b) determining interaction or binding between the polypeptide or peptide the and test substance.

A substance which interacts with the polypeptide or peptide of the invention may be isolated and/or purified, manufactured and/or used to modulate its activity as discussed.

A further aspect of the present invention provides an assay method which includes:
(a) bringing into contact a substance including a DUB polypeptide or fragment, mutant, variant or derivative thereof, a substance including a fragment of a second polypeptide or a fragment, mutant, variant or derivative of said second polypeptide; which is able to bind the DUB polypeptide; and a test compound, under conditions in which in the absence of the test compound being an inhibitor the two said substances interact; wherein said DUB polypeptide is a DUB-3, DUB-4, DUB-5, DUB-6, DUB-7, DUB-8, DUB-9, DUB-10, DUB-11 or DUB-12 polypeptide;
(b) determining interaction between said substance.

The invention will now be described further in the following non-limiting examples with reference made to the accompanying drawings in which:

FIG. 1A illustrates ClustalX alignments of the DUB deubiquitinating enzymes. The clustal shows representative members of the DUB family of deubiquitinating enzymes including both murine (DUB-1, DUB-2, DUB-2A) and human (DUB-3) members. The positions of the six conserved domains of the ubps (DHI→DHVI) are underlined, each copy of the sequence repeated in the carboxy terminus is underlined with a dotted line and the cysteine, histidine and aspartate residues that make up the active site are indicated by stars (*). The sequence accession numbers are: DUB-1→ NM₁₃ 007887; DUB-2→NM₁₃ 010089; DUB-2A→ AF393637; DUB-3→xxxxxxxx.

FIG. 1B illustrates the amino acid sequences for DUB-3, DUB-4, DUB-5, DUB-6, DUB-7, DUB-8, DUB-9, DUB-10, DUB-11 or DUB-12.

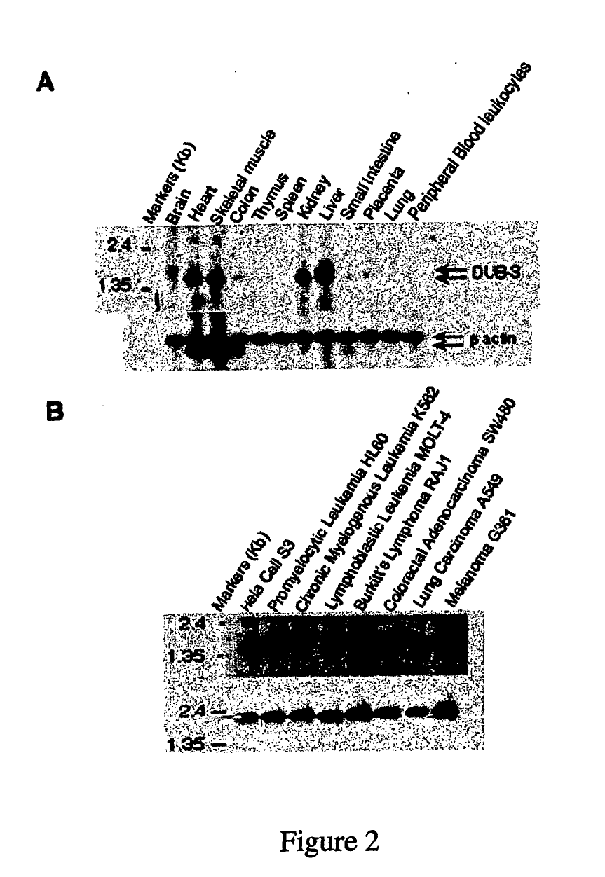

FIG. 2 illustrates Northern blot analysis of DUB expression. A) Multiple tissue Northern blot (BD Biosciences Clontech, CA, USA). Two transcripts observed, one of approximately 1.6 Kb in a number of tissues including heart, skeletal muscle, colon, kidney and liver, and a second of approximately 1.7 Kb in brain and liver; B) Tumour cell line Northern blot. Expression of the 1.6 Kb transcript observed in all the cell lines examined except K562. Expressing cell lines derived from a range of different human tumours.

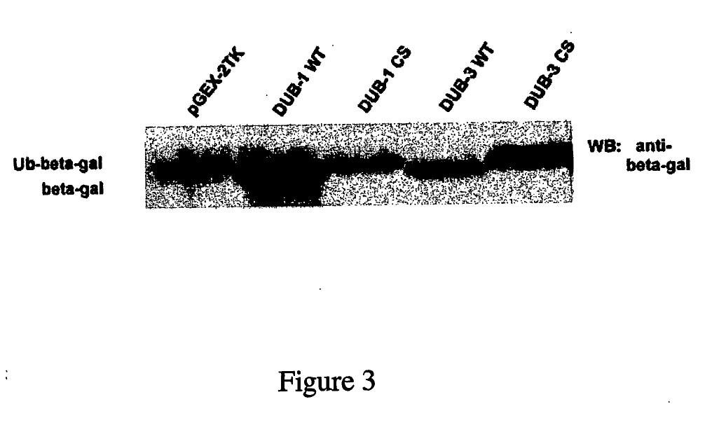

FIG. 3 illustrates DUB-3 is a functional deubiquitinating enzyme. Extracts were prepared from E. coli co-transformed with a vector expressing the ubiquitin-β-galactosidase (Ub-Met-β-gal) fusion protein as well as the plasmids pGEX-2TK (No DUB expression) (lane 1); pGEX-DUB-1 (lane 2); pGEX-DUB-1 (C60) (lane 3); pGEX-DUB-3 (lane 4); pGEX-DUB-3C89S (lane 5). Deubiquitinating activity was demonstrated for both DUB-1 and DUB-3 fusion proteins by the cleavage of the Ub-Met-β-gal fusion. DUB-1C60S (lane 3) and DUB-3C89S (lane 5) are catalytically inactive.

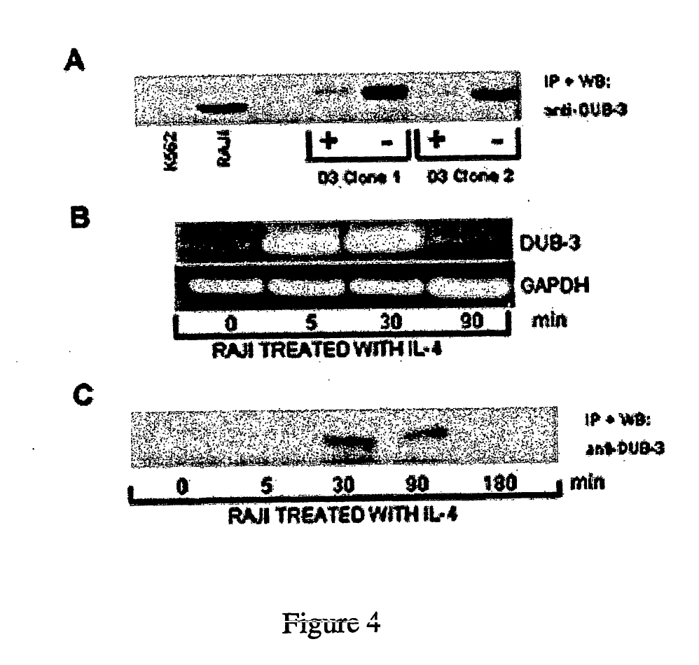

FIG. 4 illustrates Regulation of DUB protein expression. A) Protein lysates were extracted from 10⁷ cells of K562 (Chronic Myelogenous Leukemia ) and RAJI (Burkitt's lymphoma) as well as the DUB3 expressing Ba/F3 clones 1 and 2 cultured +/− tetracycline. Lysates were immunoprecipitated and immunoblotted with DUB-3 antibody; B) RAJI cells cultured in serum free medium for 4 hours before treatment with IL-4 (50U/ml) for times indicated and RT-PCR was performed; B) Protein lysates were extracted from 10⁷ RAJI cells treated as in B and immunoprecipitates were prepared and treated as in A.

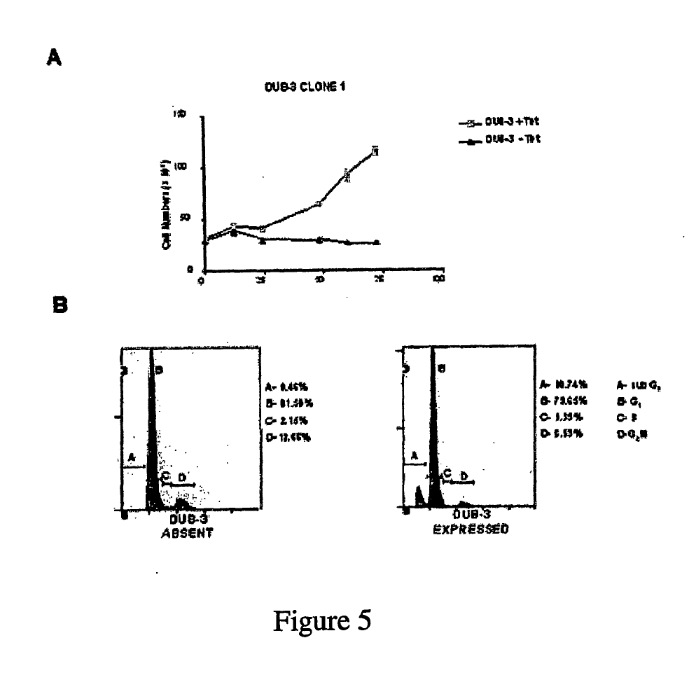

FIG. 5 illustrates DUB-3 expression slows proliferation and increases the rate of apoptosis. A) DUB3 expressing Ba/F3 cells seeded at 2×10⁵ cells per ml and cultured +/− tetracycline were analysed at 12 h intervals by trypan blue exclusion assay; B) DUB3 expressing Ba/F3 cells were stained using propidium iodide after 48 h +/− tetracycline and analysed using flow cytometry.

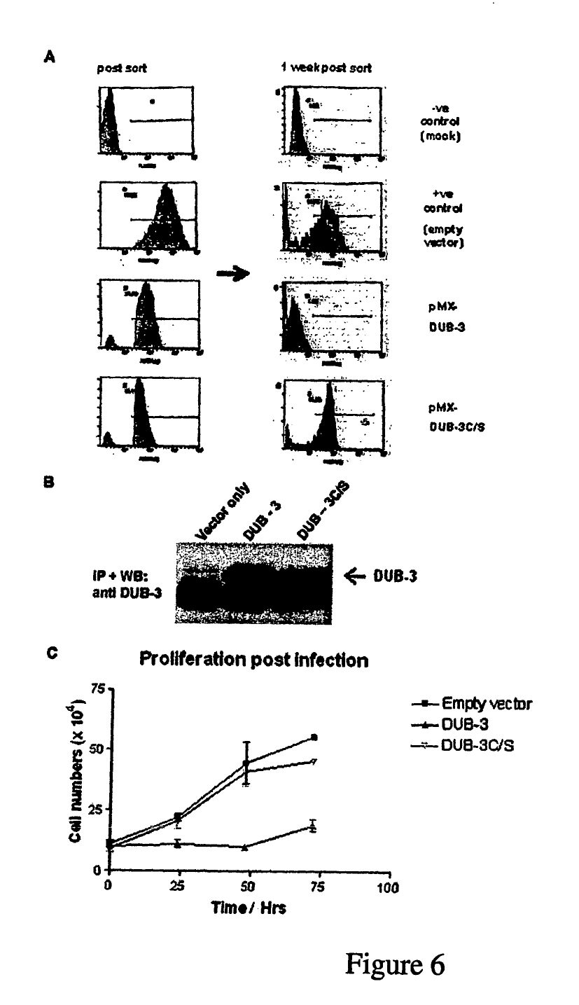

FIG. 6 illustrates infection of Ba/F3 cells with DUB-3 and DUB-3C/S expressing retroviral vectors. Supernatants from PlatE cells transfected with pMX-ires-EGFP-DUB-3, pMX-ires-EGFP-DUB-3C/S or empty vector were used to infect Ba/F3 cells: A) The Ba/F3 cells were examined at 48 h using flow cytometry for the presence of GFP. Panel 1 shows the proportion of the cell population infected post cell sorting. Panel 2 shows the proportion of cell population infected one week post sorting; B) Protein lysates were extracted from 10⁷ Ba/F3 cells infected as before were immunoprecipitated and immunoblotted with DUB-3 antibody; C) Cell populations infected and sorted as in A were cultured and the number of viable cells determined every 24 h using the trypan blue exclusion assay.

FIG. 7 shows the nucleic acid sequences (SEQ ID NO: 2) which encode the amino acid sequences for DUB-3, DUB-4,-DUB-5, DUB-6, DUB-7, DUB-8, DUB-9, DUB-10, DUB-11 and DUB-12.

FIG. 8 shows Western blots which show that DUB-3 expression down-regulates signaling through the Ras/MEK/ERK pathway. Top and middle panels show ERK and MEK phosphorylation is reduced in cells expressing DUB-3. The bottom panel shows a slower migrating form of Ras in those samples were DUB-3 is expressed.

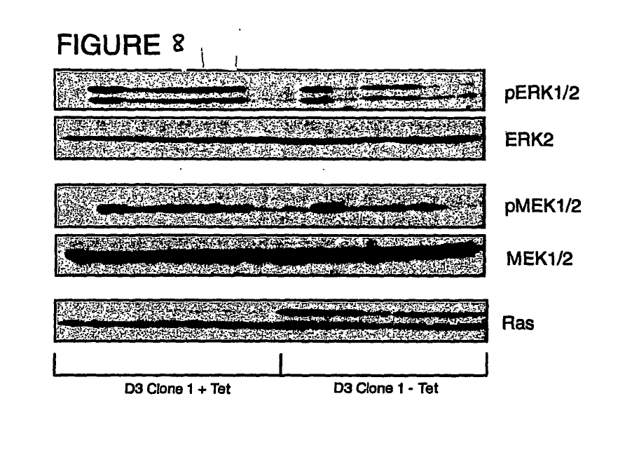

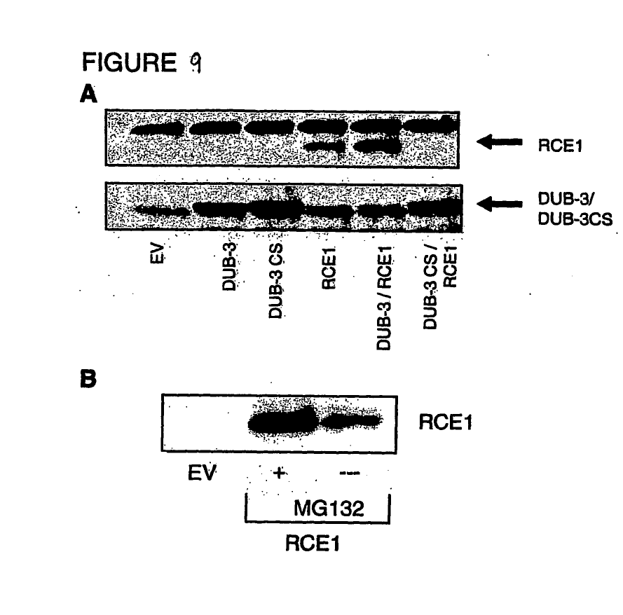

FIG. 9 shows Western blots which show that RCE1 is the substrate of DUB-3.

### EXAMPLES

### Experimental Procedures

Database Analysis

Basic sequence manipulations were performed with the Lasergene (DNAStar) suite of programs. More advanced analyses were conducted through the web-based portal NIX (http://www.hgmp.mrc.ac.uk/Registered/Webapp/nix/) at the UK HGMP Resource Centre. This provides an interface to multiple databases for simultaneous BLAST searching including trembl, swissprot, embl, est and htg and displays the collective outputs as a simplified hyper-linked graphical representation.

cDNA Cloning

RNA samples were extracted from 5×10⁶ RAJI cells using Stat-60 reagent (TEL-TEST INC, TX, USA). RT-PCR was carried out using the OneStep RT-PCR System (Qiagen, West Sussex, UK) and the primers sets:

D1→5′-CAGTGAATTCGTGGGAATGGAGGACGACTCACTCTAC -3′,
D2→5′-AGTCATCGATCTGGCACACAAGCATAGCCCTC -3′.

RT-PCR products were cloned using the Perfectly Blunt Cloning Kit pT7Blue-3 (Novagen, WI. USA) and sequenced using the BigDye Terminator v3.1 kit, the ABI Prism 3100 Genetic Analyzer (Applied Biosystems, Cheshire, UK) and the primers:

D3→5′-CTCATGTTCACTGTGGATGC-3′;
D4→5′-GATGATCTACCTGCTTGTGG-3′;
D5→5′-CGGACATTACTTCTCTTATG-3′;
D6→5′-GGACAGAAGTGATGCTAGAG-3′;
D7→5′-TTGCCAACTACATGCTGTCC-3′;
D8→5′-CTGGGTGGAGTTGTCACAAC-3′.

Northern Blotting

Total RNA was extracted from cell lines using an RNeasy midi kit (Qiagen) and quantitated by spectrophotometry. 20 μg of total RNA from each sample was subjected to electrophoresis in a 1% agarose formaldehyde gel. Gels were washed three times with water and then denatured for 30 min in 50 mM NaOH-10 mM NaCl, rinsed with 100 mM Tris HCl (pH 7.5) for 30 min, and placed in 10×SSC (1×SSC is 0.15 M NaCl plus 0.015 M sodium citrate) for 30 min. Gels were transferred overnight onto nylon membranes (Schleicher & Schleicher, N.H., USA). Following transfer, membranes were rinsed with 2×SSC and cross-linked in a UV Stratalinker 2400 (Stratagene, CA, USA). Membranes were prehybridized in 5 ml of Rapid-hyb (Amersham, Buckinghamshire, UK) and then hybridized with cDNAs labelled by random priming using the Rediprime DNA labeling system (Amersham). Following hybridization, membranes were subject to a final wash in 0.1×SSC-0.1% sodium dodecyl sulfate (SDS) at 65° C. before exposure to film.

Plasmids

DUB-3 cDNAs were tagged with the FLAG epitope at their C-termini by standard PCR based methods. Each CDNA was amplified by PCR, using a 5′ oligonucleotide containing an EcoRI site and an ATG codon as well as a 3′ oligonucleotide containing a ClaI site. The EcoRI/ClaI PCR fragment was sub-cloned between the EcoRI and ClaI sites of a modified pME18S vector in frame with the FLAG epitope. To produce the catalytically inactive DUB-3C/S mutant the cysteine residue at position 89 was changed to a serine using the Quickchange in-vitro mutagenesis kit (Stratagene). The pUHD 10-3 plasmids expressing DUB-3 were constructed by sub-cloning the DUB-3-FLAG cDNA. The pMX-ires-EGFP plasmids expressing DUB-3 and DUB-3C/S were constructed by sub-cloning the DUB-3-FLAG and DUB-3C/S-FLAG cDNAs.

Cell Culture and Transfections

The IL-3-dependent pro-B cell line Ba/F3 and Ba/F3b/tTA²⁸ were grown in RPMI 1640/10% FBS containing 10 ng/ml IL-3. RAJI and K562 cells were grown in RPMI 1640/10% FBS. PlatinumE (PlatE: derived from 293T fibroblast, kind gift from Dr T. Kitamura, University of Tokyo, Japan) and NIH3T3 cells were grown in Dulbecco's modified eagle medium/10% FBS. Transfectants of Ba/F3b/tTA cells were generated by electroporating 10⁷ cells with linearized pUHD10-3 plasmid containing DUB-3 using a Gene Pulser (Bio-rad, CA, USA ; 300 V, 960 mF) and selected using 1.2 μg/ml hygromycin. Tetracycline (4 μg/ml) was also added and replaced every 48 h. To induce expression of DUB-3/DUB-3C/S, cells were grown in the absence of tetracycline for 24 to 48 h. Transfectants of PlatE cells were generated using FuGENE 6 transfection reagent (Roche, East Sussex, UK), as specified by the manufacturer, and either empty vector or pMX-ires-EGFP that expresses the DUB-3-FLAG or DUB-3C/S-FLAG fusion proteins.

Antibody Production

A DUB-3 polyclonal antibody was obtained from Fusion Antibodies (Belfast, UK). Immunisations were performed using an affinity purified recombinant 6 HIS tagged fusion protein containing residues 378 to 530 of the DUB-3 protein.

Cell Lysis, Immunoprecipitations and Western Blotting

To produce whole cell lysates cells were washed in PBS and lysed in 50 mM Tris pH 7.4, 150 mM NaCl, 5 mM EDTA, 0.875% Brij 97, 1 mM Na₃VO_{4, 10} μg/ml leupeptin, 10 μg/ml aprotinin, and 1 mM phenylmethylsulfonyl flouride (PMSF), and centrifuged at 12 000 rpm at 4° C. for 10 minutes. Immunoprecipitations were carried out using a rabbit polyclonal DUB-3 antibody (Fusion antibodies) or the M2 anti-FLAG antibody (Sigma, MO. USA). The immunoprecipitates were then washed 5 times in lysis buffer. Cellular lysates and immunoprecipitates were separated using sodium dodecyl sulphate-polyacrylamide gel electrophoresis (SDS-PAGE), transferred onto polyvinylidene difluoride (PVDF) membranes (Millipore, MA. USA), and immunoblotted as appropriate using antibodies against either DUB-3 or FLAG. The blots were visualized by enhanced chemiluminescence (ECL, Amersham).

Deubioquitinating Assay

The deubiquitination assay, based on the cleavage of ubiquitin-β-galactosidase (ub-β-gal) fusion proteins, has been described previously³⁴. A 1590-base pair (bp) fragment corresponding to the DUB-3 ORF (amino acids 1 to 530) and an equivalent ORF containing a catalytically inactive mutant form, DUB-3C/S (C89S), were generated by PCR and inserted, in frame, into the pGEX vector in frame with the GST epitope. Ub-Met-b-galactosidase was expressed from a pACYC184-based plasmid. Plasmids were co-transformed into MC1061 Escherichia coli. Plasmid-bearing E coli MC1061 cells were lysed and analyzed by inmunoblotting with a rabbit anti-β-galactosidase antiserum (Cappel, NC, USA).

Growth Factor Station

RAJI cells were washed and incubated for 4 hours in serum free medium. The cells, remaining in serum free medium, were then treated with 100 U/ml IL-4 for the specified times before either lysates were taken for immunoprecipitation with the DUB-3 antibody or RNA samples were extracted using the Stat-60 reagent (TEL-TEST INC) for RT-PCR. RT-PCR was carried out using the OneStep RT-PCR System (Qiagen) with the following primers sets:

D1→5′-CAGTGAATTCGTGGGAATGGAGGACGACTCACTCTAC -3′,
D2→5′-AGTCATCGATCTGGCACACAAGCATAGCCCTC -3′
and
GAPDH F 5′-TGATGACATCAAGAAGGTGG-3′
GAPGH R 5′-TTACTCCTTGGAGGCCATGT -3′.

Retroviral Infection

The bicistronic vector pMX-ires-EGFP was transfected into the packaging cell line PlatE. 24 h post transfection the medium was removed and used to resuspend Ba/F3 cells at 2.5×10⁵ cells per ml (50 units/ml IL-3, 10 μg/ml polybrene Sigma). Cells were centrifuged in six well plates for 270 minutes at 2000 rpm at room temperature before the medium was replaced with RPMI 1640/5% FBS (50 units/ml IL-3). After 24 h cells were washed and re-suspended in serum free medium before sorting. Cells were sorted for the presence of GFP using Coulter EPICS ALTRA and EXPO software (Beckman Coulter, Buckinghamshire, UK).

Trypan Blue Exclusion Assay

Cells cultured for 12 h in medium lacking both IL-3 and serum were seeded at 2×10⁵ cells per ml and grown in RPMI 1640/10% FBS containing 10 ng/ml IL-3 in the presence or absence of 4 μg/ml tetracycline. After the time periods identified the numbers of viable cells determined by standard trypan blue exclusion assay using a 0.4% trypan blue stain solution (Invitrogen, Paisley, UK). Cell counts were carried out in duplicate.

Propidium Iodide (PI) Staining

To assess the cell cycle profile of the selected Ba/F3 clones they were stained with propidium iodide and analysed by flow cytometry. Briefly, cells were washed once in phosphate-buffered saline (PBS), re-suspended in a hypotonic buffer containing 0.1% nonylphenoxy polyethoxy ethanol (NP-40, Sigma), 0.1% sodium citrate, and 50 mg/ml PI (Sigma), and subjected to flow cytometry analysis using Coulter XL (Beckman Coulter).

Results

Identification of Human DUB Genes

As no human members of the DUB family of deubiquitinating enzymes were known extensive analysis of the available human EST and genomic databases was carried out using the sequences of murine DUB-1 and DUB-2 to identify potential human family members. This revealed multiple homologous genomic sequences from human chromosomes 4 and 8 (see supplementary figure) one of which was the chromosome 4 tandem repeat RS447that has previously been reported to contain USP17, a putative deubiquitinating enzyme³⁵. Using these sequences as a basis, primers were designed to allow the cloning and sequencing of full length RT-PCR products from the RAJI cell line to identify sequences expressed at the mRNA level. Several different cDNAs were observed (results not shown) the most frequently detected of which the inventors have designated DUB-3 (FIG. 1).

FIG. 1 shows an alignment of the protein sequences of the members of the DUB family of deubiquitinating enzymes including murine DUB-1, DUB-2 and DUB-2A, as well as DUB-3 as a representative human member. This alignment demonstrates the extensive homology between the human and murine members of the DUB protein family over the catalytic core of these enzymes including the previously identified conserved domains of the UBPs (DH I→DH VI) as well as the cysteine, aspartate and histidine residues conserved at their active site^{7, 8}. In addition, a 19 AA repeated sequence is highlighted (broken underline) that is present three times in DUB-2/2A, twice in DUB-1, but only once in the human DUBs. The significance of this is unclear.

Tissue Distribution

To demonstrate that DUB-3 was expressed at the mRNA level Northern blot hybridisation of poly (A)+ RNA's from multiple tissues was carried out using the full length DUB-3 CDNA as a probe (FIG. 2a). Two transcripts were observed, one of approximately 1.6 Kb in a number of tissues including heart, skeletal muscle, colon, kidney and liver, with a second transcript of approximately 1.7 Kb present in both brain and liver.

In addition the inventors carried out Northern blot hybridisation of RNA's from a number of cell lines including those derived from a range of haematopoietic tumours such as promyelocytic leukaemia, chronic myelogenous leukaemia, lymphoblastic leukaemia and Burkitt's lymphoma as well as a range of solid tumours (FIG. 2b). All but one of the cell lines examined showed high level expression of the 1.6 Kb transcript. These observations contrasted with those in the multiple tissue Northern where normal peripheral blood leukocytes and spleen (FIG. 2a) showed negligible levels of expression.

Deubiquitinating Assay

Having cloned DUB-3 it was important to demonstrate that it was an active enzyme and as a result an in vitro deubiquitination assay was performed. DUB-3 was co-expressed in E. coli cells with a ubiquitin-Met-β-galactosidase fusion protein. Activity was assessed by the cleavage of ubiquitin from the ubiquitin-Met-β-galactosidase fusion protein and visualised by immunoblotting using an anti-β-galactosidase polyclonal antibody. The results demonstrated that DUB-3 displayed deubiquitinating activity at least equivalent to that of DUB-1 and that this activity was ablated by mutating the cysteine residue (C89S) conserved within the active site which has previously been shown in other deubiquitinating enzymes to be essential for catalytic activity⁶ (FIG. 3).

Regulation of DUB Protein Expression

To facilitate studies of the endogenous protein a rabbit polyclonal antibody was developed against the carboxy-terminus of DUB-3 using a purified recombinant fusion protein containing amino acid residues 378-530, a region outside the recognised catalytic core (DHI-DHVI) (FIG. 1). The antibody was tested using Ba/F3 cells expressing a DUB-3-FLAG-tagged fusion protein from a TET-Off expression construct. The findings demonstrated that the antibody could immunoprecipitate and immunoblot the DUB-3 protein (FIG. 4a). These results were confirmed by immunoprecipitations and blotting using the anti-FLAG antibody M2 (data not shown). Having demonstrated that the DUB-3 antibody recognised over-expressed DUB-3 protein it was next important to examine whether it could recognise endogenously expressed protein. Previously the inventors had shown that the cell line RAJI expressed high levels of DUB-3 MRNA (FIG. 2 and results not shown). Therefore this cell line was chosen to test for the presence of endogenous protein. Lysates from RAJI cells were immunoprecipitated using the DUB-3 antibody and examined by Western blotting. Constitutive expression of DUB-3 protein was detected in the RAJI cell line (FIG. 4a) demonstrating that these cells expressed DUB-3 at both the mRNA and protein levels. K562 cells which did not express DUB-3 mRNA (FIG. 2b) also did not show protein expression (FIG. 4a).

DUB-1 and DUB-2 were originally identified as immediate early genes rapidly induced upon cytokine stimulation. In particular, DUB-1 has been shown to be induced by IL-3, IL-5 and GM-CSF²⁴ whilst DUB-2 was induced in response to IL-2²⁵. To determine whether or not DUB-3 behaved in a similar manner a study was carried out to assess its inducibility in response to IL-2 and IL-4 stimulation. DUB-3 MRNA and protein levels were significantly reduced by placing cells in serum free medium for 4 h prior to growth factor stimulation (FIG. 4b+c). Following stimulation the mRNA expression was determined by RT-PCR using primers capable of amplifying DUB-3 and at the protein level by immunoprecipitation and blotting using the previously characterised DUB-3 antibody. Expression of DUB-3 was induced at both the mRNA and protein level in response to IL-4 (100U/ml) (FIG. 4b +c). Up-regulation of DUB-3 mRNA was observed in samples taken as little as 5 min after IL-4 treatment and maintained for at least 30 min with the levels falling back to baseline before 90 min. In addition, to determine if particular transcripts were responsible for this induction the RT-PCR products obtained during these experiments were cloned and 10 colonies were selected for sequencing. In addition to DUB-3, several different transcripts were represented suggesting that this induction was not due to the up-regulation of a particular transcript, but to the simultaneous up-regulation of multiple DUB transcripts (data not shown). Accordingly, induction of DUB-3 protein production followed that of the mRNA with up-regulation of the protein levels being observed after 30 min, and levels not returning to baseline until after 90 min. These results suggested that human DUB-3 is a cytokine inducible immediate early gene transiently expressed upon IL-4 stimulation.

Expression Studies

Having identified DUB-3 as a cytokine inducible transiently expressed protein, the inventors next explored the functional importance of its expression. To examine this, Ba/F3 cell lines which expressed DUB-3 from a TET-Off expression system were used (FIG. 4a). To assess the effects of DUB-3 expression on proliferation, cells cultured in medium lacking IL-3 and serum for 12 h were seeded at 2×10⁵ cells per ml and cultured in RPMI 1640/10% FBS containing 10 ng/ml IL-3 in the presence and absence of tetracycline. Samples were taken every 12 h and the numbers of Viable cells determined using the trypan blue exclusion assay. The DUB-3 expressing clones examined showed a substantial reduction in their rate of proliferation when DUB-3 was expressed (FIG. 5a). The results shown were reproduced using two separate DUB-3 expressing clones and are representative of 5 experiments. In addition, it was noted that 24-48 h after the removal of tetracycline approximately 10% of the cells observed were non-viable (results not shown). This indicated that DUB-3 expression induced cell death in addition to slowing proliferation. It has been observed previously that expression of DUB-1 can result in cell cycle arrest prior to S phase²⁷. This raised the possibility that DUB-3 expression might act as a cell cycle block. To examine this in more detail, cells cultured in the presence and absence of tetracycline, were stained with propidium iodide (PI) to determine their cell cycle profile (FIG. 5b). Cells expressing DUB-3 showed a 50% reduction in the numbers of cells in S-phase and G₂M suggesting cells expressing DUB-3 may not be able to exit G₁. Moreover, when tetracycline was removed approximately 10% of cells were observed in the sub G₁ region indicating cells undergoing apoptosis. This confirmed the observations made with the trypan blue exclusion assay and indicated that expression of DUB-3 blocks proliferation and can initiate apoptosis.

To determine whether DUB-3's effect was cell type specific, Ba/F3 and NIH3T3 cells were established expressing either DUB-3 or DUB3C/S using the bicistronic vector pMX-ires-EGFP. The pMX-ires-EGFP vector expressed both the DUB-3 construct and GFP allowing expressing cells to be selected for green fluorescence. The PlatE packaging cell line was transiently transfected with either pMX-ires-EGFP or pMX-ires-EGFP containing DUB-3 or DUB-3C/S and supernatant was used to infect both Ba/F3 and NIH3T3 cells. The proportion of infected cells was then enriched by sorting cells based on GFP expression (FIG. 6a and not shown). DUB-3 and DUB-3C/S expression was confirmed by immunoprecipitation and immuno-blotting using the DUB-3 antibody (FIG. 6b and not shown). The enriched cell populations, which now had a minimum of 80% of their cells infected, were cultured for one week and the proportion of cells infected reassessed using flow cytometry. The proportion of cells expressing GFP in the populations infected with vector alone or DUB-3C/S remained within 10% of the levels following sorting. However, in both the Ba/F3 and NIH3T3 cells, the proportion of cells expressing GFP in those cell populations infected with pMX-ires-EGFP-DUB-3 dropped to <1% (FIG. 6a and not shown). These results suggested that, as previously observed, cells expressing DUB-3 were proliferating significantly slower than the uninfected cells allowing the uninfected population, which had originally only represented ˜10-20% of the population, to rapidly outgrow the DUB-3 expressing cells. To further confirm these observations populations of Ba/F3 cells were infected as before and their rate of proliferation examined following sorting using the trypan blue exclusion assay (FIG. 6c). Again the cell population infected with pMX-ires-EGFP-DUB-3 grew more slowly than the control cells. These results confirmed that DUB-3 expression blocked proliferation and indicated that it was not a cell type specific phenomenon as it occurred in both the Ba/F3 and NIH3T3 cell lines. Importantly, it was also noted that the populations of cells expressing the catalytically inactive DUB-3C/S behaved in a similar manner to the populations infected with the vector only control suggesting that the effect of DUB-3 on proliferation is dependent on its deubiquitinating activity.

To determine how DUB-3 blocks cell proliferation the inventors examined the activation of a number of intracellular signalling molecules in the presence and absence of DUB-3 expression. The results are shown in FIG. 8. Ba/F3 cells which express DUB-3 from a Tet-Off expression vector were seeded at 2×10⁵ cells per ml and cultured +/− tetracycline for 48 Hrs. These cells were then cultured —IL-3 and FCS for 4 Hrs before stimulation with 10 ng/ml IL-3. Lysates were taken at the indicated time points and analysed by Western blotting. A significant down-regulation of ERK phosphorylation was observed in cells expressing DUB-3 indicating that the Ras/MEK/ERK signalling pathway was down-regulated (FIG. 8). Subsequently the inventors have demonstrated that MEK phosphorylation is also down-regulated indicating that DUB-3 influences this pathway upstream of both ERK and MEK (FIG. 8). Finally, the inventors have shown that in the presence of DUB-3 expression a slower migrating form of Ras is observed (FIG. 8). This slower migrating form is indicative of a disruption of Ras processing and indicates that DUB-3 acts to down-regulate the Ras/MEK/ERK pathway by disrupting the processing of Ras.

To determine how DUB-3 could disrupt the processing of Ras the inventors examined the affect of DUB-3 expression on a number of enzymes involved in this process. 293T cells were transfected with constructs expressing the proteins indicated and lysates were taken after 24 Hrs. These were then analysed by Western blotting to determine the expression of RCE1 (FIG. 9A top panel) and DUB-3/DUB-3CS (FIG. 9A bottom panel). 293T cells were transfected with a construct expressing RCE1 and after 24 Hrs these transfections were either treated with 1 mM MG132 (an inhibitor of the proteasome) or mock treated before lysates were taken. These were then analysed by Western blotting to determine the expression of RCE1 (FIG. 9B). Whilst other components of this pathway were unaffected the inventors observed that the enzyme RCE1 (Ras converting enzyme 1) was proteasomally regulated (FIG. 9b) and co-expression of RCE1 and DUB-3 resulted in the levels of RCE1 increasing significantly (FIG. 9A). These results indicate that RCE1 protein levels are regulated by degradation through the ubiquitin-proteasome pathway and that expression of DUB-3 blocks this degradation. This would indicate that the substrate of the deubiquitinating enzyme DUB-3 is RCE1 and that it blocks its targeting to the proteasome by removing the ubiquitin chains which target it for degradation. RCE1 is involved in the processing of multiple CAAX motif containing proteins in addition to Ras (Rho, Rap, CDC42) and it can therefore be surmised that DUB-3 expression could influence their activation through its effects on RCE1.

Discussion

In this study the inventors report the cloning of DUB-3, a human member of the DUB family of deubiquitinating enzymes, and show its expression at the mRNA level in a range of tissues and cell lines. The inventors also demonstrate that DUB-3, which the inventors have shown to be an active deubiquitinating enzyme, is induced at both the mRNA and protein level in response to IL-4 stimulation and that constitutive expression of DUB-3 blocks proliferation and leads to an increase in the number of apoptotic cells observed.

Prior to this report no human members of the DUB family had been identified. However, several studies had indicated that the murine DUBs may be important for the regulation of proliferation and survival in immune cells. DUB-1 (IL-3, IL-5, GM-CSF)²⁴ and DUB-2 (IL-2)²⁵ are haematopoietic specific immediate early genes induced in response to cytokine stimulation and over-expression of DUB-1 results in a cell cycle arrest prior to S-phase²⁷. Moreover, the inventors have recently demonstrated that DUB-2 expression can markedly inhibit apoptosis induced in response to growth factor removal²⁸.

Our initial database analyses identified a large number of highly similar sequences showing extensive homology to the murine DUBs especially within their catalytic domains. The observation of so many sequences demonstrating high levels (>95%) of identity, in conjunction with the identification of one of these sequences as a repeat (RS447), indicated that these sequences form part of a repetitive unit present on human chromosomes 4 and 8. This also suggested many of these sequences could represent non-functional pseudogenes. Therefore, to identify functional genes, the inventors set about cloning those sequences expressed at the mRNA level. A number of different transcripts were observed in the RAJI cell line, and in response to IL-4 stimulation, suggesting that there are multiple functional genes. However, as the high levels of similarity suggests these transcripts encode functionally redundant proteins the inventors chose the most frequently observed transcript, DUB-3, for use in all further experiments.

The relationship between DUB-3 and the previously identified murine genes is unclear. The levels of homology observed, as well as DUB-3's induction in response to IL-4 would suggest it is a member of the DUB family of enzymes. However, in contrast to DUB-1, DUB-2 and DUB-2A, previously shown to be primarily expressed in haematopoietic cells^{24, 25, 26}, DUB-3 showed little detectable expression in peripheral blood leukocytes and the spleen. Therefore, DUB-3 appears not to be haematopoietic specific although its induction in response to IL-4, as well as its expression in a number of leukaemia and lymphoma cell lines, would suggest it is expressed in leukocytes in a highly regulated manner.

DUB-3's regulation by IL-4 suggested that like its murine counterparts it may regulate immune function. IL-4 has several roles in the immune system including, directing Th2 development and expansion³⁶, regulating B-cell growth and differentiation³⁷, secretion of IgE and IgG4³⁸ and mast cell growth³⁹. It could therefore be hypothesised that DUB-3 expression may well play a role in all of these processes. DUBs are not the only UBPs that play a role in the immune system. UBP43 (Usp18) has been shown to be induced in response to IFN (type 1) and lipopolysaccharide (LPS) and shown to cleave ISG15, a ubiquitin-like protein, from ISG-conjugated substrates⁴⁰. ISG15 is also strongly induced in response to IFN and LPS and it has been proposed that ISG15 and UBP43 may combine to regulate signalling through the JAK-STAT pathway possibly allowing them to regulate some immune responses⁴¹. In addition, CYLD, a deubiquitinating enzyme has recently been demonstrated to regulate activation of the NF-κB pathway by TNFR family members⁴².

The observation that expression of DUB-3 blocks proliferation and induces apoptosis indicated that like the murine DUBs it can influence proliferation and survival. It also suggests that DUB-3 acts to block the degradation of a protein involved in the negative regulation of proliferation. In particular the failure of many cells to exit G₁ would suggest that DUB-3 blocks degradation of a protein involved in the regulation of the G₁S transition. Numerous studies have previously linked the ubiquitin-proteasome system to the control of proliferation and the cell cycle. In particular, the E3 ligase SCF^{skp2} has been shown to target the cyclin dependent kinase inhibitor p27^{Kip1} for degradation and the levels of skp2 fluctuate in a cell cycle dependent manner to reduce p27^{Kip1} levels and allow the G1/S transition. Elevated levels of skp2 have also been reported in tumours where it is thought to promote tumour growth by degrading p27^{Kip1}, a known tumour suppressor⁴³. Also, a number of deubiquitinating enzymes have been implicated in the control of the cell cycle. DUB-1 expression results in a block in the cell cycle prior to S-phase²⁷ and expression of a mutant form of the deubiquitinating enzyme Ubp-M, which deubiquitinates histone H2A, has been shown to result in a cell cycle block which eventually leads to apoptosis⁴⁴

There is also growing evidence that the ubiquitin-proteasome system plays an important role in the regulation of apoptosis. Numerous molecules important for apoptosis have been shown to be proteasomally regulated such as p53, components of the NF-κB pathway and Bcl-2 family members³. Moreover, some members of the inhibitor of apoptosis (IAP) proteins have been shown to demonstrate E3 ligase activity⁴⁵. Deubiquitinating enzymes have also been linked to apoptosis. The HAUSP (USP7) deubiquitinating enzyme, which acts upon p53, has been shown to be targeted for a caspase-3 dependent proteolytic cleavage upon the initiation of apoptosis in thymocytes⁴⁶. Also, over-expression of UBP41 has been shown to result in apoptosis⁴⁷. It is therefore possible that DUB-3 may initiate apoptosis by blocking the degradation of pro-apoptotic molecule. However, the observation that DUB-3 expression may prevent the G₁S transition would suggest that the apoptosis observed is induced in response to deregulation of the cell cycle. The phenotype demonstrated upon DUB-3 expression raised some questions in reference to a number of our other observations. High level expression of DUB-3 had been observed in a range of rapidly proliferating cell lines even though constitutive expression of DUB-3 blocks proliferation. However, RAJI cells cultured in the absence of serum for 4 h exhibited dramatically lower levels of DUB-3 expression suggesting it is expressed in a cell cycle dependent manner. This indicated that DUB-3 may only be present for a short period during the cell cycle, negating any affect on proliferation. Also, expression of DUB-3 was induced in response to IL-4. In contrast to DUB-3, the best characterised functions of IL-4 are associated with promoting growth and survival. However, DUB-3 is only transiently expressed in response to IL-4 again possibly negating any effect on proliferation. Also, in addition to its better characterised functions several reports have demonstrated that IL-4 treatment results in reduced proliferation in a number of different tumour cell lines^{48, 49, 50, 51} and in one study this was also associated with the observation of an increased number of apoptotic cells⁵¹. This could suggest that the expression of DUB-3 upon IL-4 stimulation is responsible for the growth inhibition observed in these studies and that the function of IL-4 and therefore DUB-3 may be cell type specific.

In conclusion, the inventors have identified a human DUB deubiquitinating enzyme (DUB-3) which is induced in response to IL-4 and expressed in a range of tissues and cell lines. The inventors have also demonstrated that constitutive expression of DUB-3 blocks proliferation and increases the rate of apoptosis. This suggests that like its murine counterparts DUB-3 may well function to regulate immune responses by influencing cell proliferation and survival. The inventors have found that DUB-3 downregulates the Ras/MEK/ERK signalling pathway and that RCE1 is a DUB-3 substrate.

Unless the context demands otherwise, reference to DUB-3 in this discussion encompasses DUB-4, DUB-5, DUB-6, DUB-7, DUB-8, DUB-9, DUB-10, DUB-11 and DUB-12.

The results obtained with DUB-3 support the uses described herein for any one of DUB-4, DUB-5, DUB-6, DUB-7, DUB-8, DUB-9, DUB-10, DUB-11 or DUB-12.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

1) DeSalle, L. M., and Pagano, M. (2001) FEBS Lett. 490, 179-189
2) Muratani, M., and Tansey, W. P. (2003) Nat. Rev. Mol. Cell. Biol. 4, 192-201
3) Jesenberger, V., and Jentsch, S. (2002) Nat. Rev. Mol. Cell. Biol. 3, 112-121
4) Katzmann, D. J., Odorizzi, G., and Emr, S. D. (2002) Nat. Rev. Mol. Cell. Biol. 3, 893-905
5) Donaldson, K. M., Yin. H. Gekakis, N., Supek, F., and Joazeiro, C. A. (2003) Curr Biol. 13, 258-62
6) Weissman, A. M. (2001) Nat. Rev. Mol. Cell. Biol. 2, 169-178
7) D'Andrea, A., and Pellman, D. (1998) Crit. Rev. Biochem. Mol. Biol. 33, 337-352
8) Baek. K.-H. (2003) Exp. Mol. Med. 35, 1-7
9) Verma, r., Aravind, L., Oania, R., McDonald, W. H., Yates III, J. R., Koonin, E. V., and Deshaies, R. J. (2002) Science 298, 611-615
10) Evans, P. C., Smith, T. S., Lai, M-J., Williams, M. G., Burke, D. F., Heyninck, K., Kreike, M. M., Beyaert, R., Blundell, T. L., and Kilshaw, P. J. (2003) J. Biol. Chem. 278, 23180-23186
11) Huang, Y., Baker, R. T., and Fischer-Vize, J. A. (1995) Science. 270, 1828-1831
12) Pantaleon, M., Kanai-Azuma, M., Mattick, J. S., Kaibuchi, K., Kaye, P. L., and Wood, S. A. (2001) Mech. Dev. 109, 151-160
13) Moazed, D., and Johnson, D. (1996) Cell. 86, 667-677
14) Henchoz, S., De Rubertis, F., Pauli, D., and Spierer, P. (1996) Mol. Cell. Biol. 16, 5717-5725
15) Li, M., Chen, D., Shiloh, A., Luo. J. Nikolaev. A. Y., Qin, J. and Gu, W. (2002) Nature. 416, 648-653
16) DeSalle, L. M., Latres, E., Lin, D., Graner, E., Montagnoli, A., Baker, R. T., Pagano, M., and Loda, M. (2001) Oncogene. 20, 5538-5542
17) Blanchette, P., Gilchrist, C. A., Baker, R. T., and Gray, D. A. (2001) Oncogene. 20, 5533-5537
18) Taya, S., Yamamoto, T., Kanai-Azuma, M., Wood, S. A., and Kaibuchi, K. (1999) Genes Cells. 4, 757-767
19) Jensen, D. E., Proctor, M., Marquis, S. T., Gardner, H. P., Ha, S. I., Chodosh, L. A., Ishov, A. M., Tommerup, N., Vissing, H., Sekido, Y., Minna, J., Borodovskv, A., Schultz. D. C., Wilkinson, K. D., Maul, G. G., Barley, N., Berger, S. L., Prendergast, G. C., Rauscher, F. J. 3rd. (1998) Oncogene. 16, 1097-1112
20) Li, Z. B., Na, X., Wang, D. K., Schoen, S. R., Messing, E. M., and Wu, G. (2002) J. Biol. Chem. 277, 4656-4662
21) Nakamura, T., Hillova, J., Mariage-Samson, R., Onno. M., Huebner, K., Cannizzaro, L. A., Boahosian-Sell, L., Croce, C. M., and Hill, M. (1992) Oncogene. 7, 733-741
22) Gupta, K., Chevrette, M., and Gray, D. A. (1994) Oncogene. 9, 1729-1731
23) Gray, D. A., Inazawa, J., Gupta, K., Wong, A., Ueda, R., and Takahashi, T. (1995) Oncogene. 10, 2179-2183
24) Zhu, Y., Os Pless, M., Inhom, R., Mathey-Prevot, B., and D'Andrea, A. D. (1996) Mol. Cell. Biol 16, 4808-4819
25) Zhu, Y., Lambert, K., Corless, C., Copeland, N. G., Gilbert, D. J., Jenkins, N. A. and D'Andrea, A. D. (1997) J. Biol. Chem. 272, 51-57
26) Baek, K., Mondoux, M. A., Jaster, R., Fire-Levin, E., and D'Andrea, A. (2001) Blood. 98, 636-642
27) Zhu, Y., Carroll, M., Papa, F. R., HochStrasser, M., and D'Andrea, A. (1996) Proc. Natl. Acad. Sci. USA. 93, 3275-3279
28) Migone, T., Humbert, M., Rascle, A., Sanden, D., D'Andrea, A., and Johnston, J. A. (2001) Blood. 98, 1935-1941
29) Mulloy, J. C., Migone, T. S., Ross, T. M., Ton, N., Green, P. L., Leonard, W. J., and Franchini, G. (1998) J. Virol. 72, 4408-4412
30) Watanabe, T. (1997) Int. J. Hematol 66, 257-278
31) Kawano, F., Tsukamoto, A., Satoh, M., Sanada, I., Shido, T., Suzushima, H., Asou, N., and Takatsuki, K. (1992) Leukemia. 6, 66-67
32) Bromberg, J. (2002) J. Clin. Invest 109, 1139-1142
33) Ward, A. C., Touw, I., and Yoshimura, A. (2001) Blood. 95,19-29
34) Papa, F. R., and Hochstrasser, M. (1993) Nature. 366, 313-319
35) Gondo, Y., Okada, T., Matsuyama, N., Saitoh, Y., Yanagisawa, Y., and Ikeda, J. (1998) Genomics. 54, 39-49.
36) O'Garra, A., and Arai, N. (2000) Trends. Cell Biol. 10, 542-550
37) Howard, M., Farrar, J., Hilfiker, M., Howard, M., Farrar, J., Hilfiker, M., Johnson, B., Takatsu, K., Hamaoka, T., and Paul WE. (1982) Proc. Natl. Acad. Sci. USA. 85, 6107-6111
38) Pene, J., Rousset, F., Briere, F., Chretien, I., Bonnefoy, J. Y., Spits, H., Yokota, T., Arai, N., Arai, K., Banchereau, J., et al. (1988) Proc. Natl. Acad. Sci. USA. 85, 6880-6884
39) Madden, K. B., Urban, J. F., Ziltener, H. J., Schrader. J. W., Finkelman, F. D., and Katona, I. M. (1991) J. Immunol 147,1387-1391
40) Malakhov, M. P., Malakhova, O. A., Kim, K. I., Ritchie, K. J., and Zhang, D. E. (2002) J. Biol. Chem. 277, 9976-9981
41) Malakhova, O. A., Yan, M., Malakhov, M. P., Yuan, Y., Ritchie, K. J., Kim, K. I., Peterson, L. F., Shuai, K., and Zhang, D. E. (2003) Genes Dev. 17, 455-460
42) Trompouki, E., Hatzivassiliou, E., Tsichritzis, T., Farmer, H., Ashworth, A., and Mosialos, G. (2003) Nature 424, 738-739
43) Bloom, J., and Pagano, M. (2003) Semin. Cancer Biol. 13, 41-47
44) Cai, S-H., Babbitt, R. G., and Marches, V. T. (1999) Proc. Natl. Acad. Sci. USA. 96, 2828-2833
45) Yang, Y., Fang, S., Jensen, J. P., Weissman, A. M., and Ashwell, J. D. (2000) Science. 288, 874-877
46) Vugmeyster, Y., Borodovsky, A., Maurice, M. M., Maehr, R., Furman, M. H., and Ploegh, H. L. (2002) Mol. Immunol. 39, 431-441
47) Gewies, A., and Grimm, S. (2003) Cancer Res. 63, 682-688
48) Toi, M., Bicknell, R., and Harris, A. L. (1992) Cancer Res. 55, 275-279
49) Obiri, N. I., Hillman, G. C., Haas, G. P., Sud, S., and Puri, R. K. (1993) J. Clin. Invest. 91, 88-93
50) Topp, M.S., Papadimitriou, C. A., Eltelbach, F., Oelmann, E., Koehler, B., Oberberg, D., Von Marschall, Z., Reufi, B., Stein, H., Thiel, E., et al. (1995) Cancer Res. 55, 2173-2176
51) Gooch, J. L., Lee, A. V., and Yee, D. (1998) Cancer Res. 58, 4199-4205.

1. An isolated polypeptide comprising the amino acid sequence shown as DUB-3 (SEQ ID NO: 1), DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11 (SEQ ID NO: 11) or DUB-12 (SEQ ID NO: 12) in FIG. 1 , or a variant or fragment thereof.

2. The isolated polypeptide according to claim 1, wherein the isolated peptide is a variant and wherein the variant does not include the RS447 sequence (Accession no D38378).

3. The isolated polypeptide according to claim 1, wherein the polypeptide comprises the amino acid sequence shown as DUB-3 (SEQ ID NO: 1), DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11 (SEQ ID NO: 11) or DUB-12 (SEQ ID NO: 12) in FIG. 1.

4. The isolated polypeptide according to claim 1, wherein the polypeptide consists of the amino acid sequence shown as DUB-3 (SEQ ID NO: 1), DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11 (SEQ ID NO: 11) or DUB-12 (SEQ ID NO: 12) in FIG. 1.

5. The isolated polypeptide according to claim 3, wherein the polypeptide comprises the amino acid sequence shown as DUB-3 (SEQ ID NO: 1).

6. The isolated polypeptide according to claim 5, wherein the polypeptide consists of the amino acid sequence shown as DUB-3 (SEQ ID NO: 1).

7. An isolated nucleic acid molecule encoding a polypeptide which includes the amino acid sequence shown as DUB-3 in FIG. 1 (SEQ ID NO: 1) DUB-4 (SEQ ID NO: 4), DUB-5 (SEQ ID NO: 5), DUB-6 (SEQ ID NO: 6), DUB-7 (SEQ ID NO: 7), DUB-8 (SEQ ID NO: 8), DUB-9 (SEQ ID NO: 9), DUB-10 (SEQ ID NO: 10), DUB-11(SEQ ID NO: 11) or DUB-12 (SEQ ID NO: 12) of FIG. 1 or a variant or fragment thereof.

8. The isolated nucleic acid according to claim 7, wherein the nucleic acid molecule comprises the nucleotide sequence shown in FIG. 7 (SEQ ID NO: 2) or a variant or fragment thereof.

9. The isolated nucleic acid molecule according to claim 7 or claim 8, wherein the nucleic acid molecule consists of the nucleotide sequence shown in FIG. 7 (SEQ ID NO: 2).

10. The isolated nucleic acid molecule according to claim 7, wherein the nucleic acid molecule consists of the nucleotide sequence shown in FIG. 7 as DUB-4 (SEQ ID NO: 14), DUB-5 (SEQ ID NO: 15), DUB-6 (SEQ ID NO: 16), DUB-7 (SEQ ID NO: 17), DUB-8 (SEQ ID NO: 18), DUB-9 (SEQ ID NO: 19), DUB-10 (SEQ ID NO: 20), DUB-11 (SEQ ID NO: 21) or DUB-12 (SEQ ID NO: 22).

11. A specific binding member which binds to a polypeptide according to claim 1.

12. A method for identifying the presence of a polypeptide according to claim 1 in a biological sample, said method including the steps:
a) bringing said biological sample into contact with a specific binding member of claim 11;
b) determining binding of said specific binding member to said sample, wherein binding of said specific binding member is indicative of the presence of said polypeptide in said sample.

13. A method of determining susceptibility to cancer in a patient, said method comprising the steps:
a) providing a biological sample from a patient,
b) bringing said biological sample into contact with a specific binding member of claim 11, and
c) detecting binding of the specific binding member to said sample, wherein binding of said specific binding member is indicative of susceptibility to cancer.

14. A method of determining susceptibility to cancer in a patient, said method comprising the steps:
a) providing a biological sample from said sample,
b) determining the presence of a DUB-3, DUB-4, DUB-5, DUB-6, DUB-7, DUB-8, DUB-9, DUB-10, DUB-11 or DUB-12 nucleic acid or a fragment thereof in said sample, wherein the presence or presence above a predetermined minimum of said nucleic acid or fragment thereof is indicative of a predisposition to cancer.

15. The method according to claim 14, wherein the method comprises the steps:
a) providing a biological sample from a patient,
b) bringing said biological sample into contact with a nucleic acid molecule which is capable of hybridising, under stringent conditions, to a nucleic acid molecule according to claim 7, under conditions which allow hybridisation of substantially complementary nucleic acids, and
c) detecting hybridisation of nucleic acids.

16. A method of diagnosis of a cancer in a patient, said method comprising the steps:
a) providing a biological sample from said sample,
b) determining the presence or relative amount of a DUB-3, DUB-4, DUB-5, DUB-6, DUB-7, DUB-8, DUB-9, DUB-10, DUB-11 or DUB-12 nucleic acid or fragment thereof in said sample, wherein the presence or presence above a predetermined minimum of said nucleic acid or fragment thereof is indicative of cancer.

17. The method of claim 16, said method comprising the steps:
a) providing a biological sample from a patient,
b) bringing said biological sample into contact with a nucleic acid molecule which is capable of hybridising, under stringent conditions, to a nucleic acid molecule according to claim 7, under conditions which allow hybridisation of substantially complementary nucleic acids, and
c) detecting hybridisation of nucleic acids, wherein detection of hybridisation is indicative of the presence of cancer.

18. A method of treatment of cancer in a patient in need thereof, said method comprising the step of administering an agent which modulates the expression of at least one of DUB-3, DUB-4, DUB-5, DUB-6, DUB-7, DUB-8, DUB-9, DUB-10, DUB-11, and DUB-12, in the cancer cells.

19. The method according to claim 18, wherein the agent enhances expression of at least one of DUB-3, DUB-4, DUB-5, DUB-6, DUB-7, DUB-8, DUB-9, DUB-10, DUB-11, and DUB-12.

20. The method according to claim 18, wherein the agent inhibits expression of at least one of DUB-3, DUB-4, DUB-5, DUB-6, DUB-7, DUB-8, DUB-9, DUB-10, DUB-11, and DUB-12.

21. An assay method for identifying an agent having anti-proliferative activity, said method comprising the steps of:
a) bringing a candidate agent into contact with a polypeptide according to claim 1; and
b) determining interaction or binding between the candidate agent and the polypeptide.

22. (canceled)

23. (canceled)

24. (canceled)

25. The method according to claim 13, wherein the cancer is a haematopoietic cancer, lung cancer, skin cancer, small intestinal cancer or thymus cancer.

26. The method according to claim 16, wherein the cancer is promyelocytic leukaemia, chronic myelogenous leukaemia, lymphoblastic leukaemia, Burkitt's lymphoma, cancer of the lung or cancer of the spleen.

27. The method according to claim 16, wherein the cancer is a cancer associated with increased expression of one or more CAAX motif containing proteins.

28. The method according to claim 16, wherein the cancer is a cancer associated with increased expression of Ras.

29. A method of modulating the concentration of Ras converting enzyme 1 (RCE1) in a cell, said method comprising the step of administering an agent which modulates the expression of any one of DUB-3 to DUB-12 in the cells.

30. A method of modulating the concentration of CAAX motif containing proteins in a cell, said method comprising the step of administering an agent which modulates the expression of any one of DUB-3 to DUB-12, preferably DUB-3, in the cells.

31. A method of treatment of cancer in a patient in need thereof, said method comprising the step of administering an agent which increases the expression of any one of DUB-3 to DUB-12 to said patient, wherein the cancer is a cancer associated with increased expression of a CAAX motif containing protein.

32. (canceled)

33. The method of claim 31 wherein the CAAX motif containing protein is Ras.

34. The method according to claim 18, wherein the agent is a polypeptide according to claim 1.

35. The method of claim 31, wherein the agent is DUB-3.

36. The method according to claim 18, wherein the cancer is promyelocytic leukaemia, chronic myelogenous leukaemia, lymphoblastic leukaemia, Burkitt's lymphoma, cancer of the lung or cancer of the spleen.

37. The method according to claim 18, wherein the cancer is a cancer associated with increased expression of one or more CAAX motif containing proteins.

38. The method according to claim 18, wherein the cancer is a cancer associated with increased expression of Ras.

39. The method according to claim 18, wherein the agent is a nucleic acid according to claim 7.

40. The method according to claim 18, wherein the agent is a specific binding member according to claim 1.
